(19) United States
(12) Patent Application Publication
(10) Pub. No.: US 2024/0094218 A1
(43) Pub. Date: Mar. 21, 2024

(54) ENHANCED HYBRIDOMA GENERATION

(71) Applicant: AMGEN INC., Thousand Oaks, CA (US)

(72) Inventors: Karen RICHMOND, Delta (CA); Agnieszka KIELCZEWSKA, Vancouver (CA); Ole OLSEN, Vancouver (CA)

(73) Assignee: AMGEN INC., Thousand Oaks, CA (US)

(21) Appl. No.: 18/275,856

(22) PCT Filed: Feb. 4, 2022
(86) PCT No.: PCT/US2022/015282

§ 371 Date: Aug. 4, 2023

Related U.S. Application Data
(60) Provisional application No. 63/146,135, filed on Feb. 5, 2021.

(51) Int. Cl. G01N 33/68; C07K 16/28; C12N 5/00; C12N 5/16

(52) U.S. Cl. CPC G01N 33/6845; C07K 16/28; C12N 5/0087; C12N 5/163; C07K 2317/33; C07K 2317/92

(57) ABSTRACT

Provided herein are methods of generating hybridomas and related methods of producing antigen-specific antibodies. In exemplary embodiments, the method comprises (a) preparing an enriched population of IgG-positive (IgG+) memory B cells from cells obtained from secondary lymphoid organs of one or more immunized non-human animals, wherein (i) less than or about 10% of the enriched population are IgM-positive (IgM+) B cells and/or (ii) the ratio of the IgG+ memory B cell count to IgM+ B cell count of the enriched population is greater than about 0.5, optionally, greater than about 1 or greater than about 2, (b) bulk-culturing the enriched population to obtain an expanded population; and (c) fusing cells of the expanded population with myeloma cells to obtain hybridomas. In exemplary aspects, the hybridomas obtained represent at least 10% or at least 15% of the IgG+ memory B cell repertoire produced by the immunized animals.

### CROSS REFERENCE TO RELATED APPLICATION

This application claims priority to U.S. Provisional Application No. 63/146,135, filed Feb. 5, 2021, the entire contents of which is incorporated herein by reference.

### BACKGROUND

Therapeutic antibodies constitute a predominant class of drugs, many of which are derived from in vivo immunization platforms including human antibody locus transgenic animals, and rely on the capture of specific B cell clones activated in response to antigen stimulation (Lu et al., Journal of Biomedical Science (2020) volume 27, Article number: 1). Antibodies generated in an in-vivo immune response to immunization in rodents can be captured through immortalization of the B cell population from immune tissues, which are predominantly located within the germinal centers (GC) of spleen and lymph nodes, but are also found in bone marrow, in mucosa-associated lymphoid tissue (MALT) and circulating in the blood. The process of immortalization, known as hybridoma generation, produces hybrid cells that express a membrane-bound clonal B cell receptor (BCR) as well as produce a secreted form of the same antibody clonotype. As the hybrids are continually dividing and secreting antibody, clones of required specificities can be identified and characterized, without concern for loss of the clone or lack of antibody material for testing. Established hybrids are typically robust and, when kept under selective pressure, continue to secrete antibody. Hybrids respond well to cycles of freeze-thaw and can survive decades of storage in liquid nitrogen. With the advent of Koehler and Milstein's hybridoma generation in 1975, the immortalization of B cells through the fusion to myeloma cells became the widely used method for antibody discovery from rodent species, most often mouse (G. Köhler & C. Milstein. Continuous cultures of fused cells secreting antibody of predefined specificity. Nature (1975) 256, 495-497).

The in-vivo generation of antigen-specific B cells for immortalization and the timing of immortalization, are both important aspects of hybrid generation. Typically, a response is raised in rodents over several weeks or months with repeated rounds of immunization. In response to repeated antigen exposure, B cells expressing a BCR which recognizes antigen, are stimulated to undergo isotype switching to express IgG, and to form GC within the secondary lymphoid organs (Akkaya et al., Nat Rev Immunol (2020) 20, 229-238). Within the GC, through cycles of somatic hypermutation (SMH) of the antigenic determinant and selection for higher affinity variants, the B cells mature and differentiate towards memory cells or plasma cells (Lau et al., Current Opinion in Immunology (2019); 63:29-34). Memory cells express high levels of B220/CD45R and IgG on the surface, but do not secrete antibody. When fully differentiated, the memory cell is small and in a quiescent state but has great potential to proliferate in response to stimulation by its cognate antigen or by polyclonal activation. The clonal diversity in the memory cell pool is high. In contrast, the plasma cell is a highly activated large and blasting B cell, secreting copious amount of antibody. Both surface IgG and B220/CD45R are down regulated. CD138 and TACI become highly expressed on the surface of the plasma cell (Tellier et al., Eur J Immunol 47(8): 1276-1279 (2017)). As the plasma cell terminally differentiates, the ability to divide is lost. The plasma cell has a short lifespan of a few days to weeks unless sequestered in highly specialized long-term survival niches. The diversity of the plasma cell compartment is low; the in-vivo selection process having focused in on a relatively small number of high affinity clones to mount an effective and rapid immune response to antigen.

While hybridoma generation is capable of sampling the immune repertoire, the process is highly inefficient, with successful immortalization rates in the range of 0.01%-0.001%. This inefficiency of fusion can be counteracted by increasing the number of cells for fusion using larger immunization cohorts or by working with larger animals such as rats. In view of the foregoing, there is a need for methods for enhanced hybridoma generation.

### SUMMARY

The present disclosure provides enhanced hybridoma generation (EHG) methods which provide benefits over those known in the art by virtue of the removal of IgM-positive (IgM+) B cells, the large scale, the high efficiency, and the specificity of the repertoire of the hybridomas produced. Advantageously, the presently disclosed EHG methods provide an enrichment for IgG-positive (IgG+) memory B cells with efficient bead-based removal of IgM+ B cells. IgM class-switches in culture to IgG+ B cells and dilute out the IgG+Ag+ memory B cell population with irrelevant or low affinity B cell clones. IgM+ B cells divide faster than IgG+ B cell clones. By removing the vast majority of the IgM+ B cells before culture and then culturing the IgG+ B cell enriched population (to allow them to make hundreds of clonal copies before fusion), a large portion of the clones of interest are immortalized. The result is a large hybridoma pool that facilitates the identification of rare Ag+ clones. The presently disclosed EHG methods provide a large-scale bulk culture. Liters of bulk cultures with hundreds of thousands of highly selected IgG+ memory B cells enables large scale deep repertoire mining of larger numbers of animals Additionally, in the prior art methods, only a very small number of antigen-binding clones are identified whereas the EHG of the present disclosure routinely identifies hundreds to thousands of unique binders. The EHG methods of the present disclosure also comprise B cell immortalization before identification of antigen binding clones. Hybridoma supernatant is screened. This order represents a reversed order of the steps of prior art methods (see, e.g., Steenbakkers et al., J Immunol Methods 152 (1): 69-77 (1992)) where B cells are screened for antigen-binding before they are immortalized. The order of immortalization followed by identification of antigen binding clones of the present invention enables high efficiency of identifying antigen-binding clones. In the presently disclosed methods, an unlimited supply of cell supernatant (SN) is available for screening and characterization after fusion. The assay sensitivity at time of SN screening is not limiting. Hybrid cultures can be grown to high concentrations of antibody. In the presently disclosed EHG methods, the ability to identify antigen-binding clones is not limited by B cell antibody secretion rate and rare binders can be identified by deep mining the hybrid pool. The deep mining by large scale liquid handling plating and Ag+ FACS sorting can be carried out.

Accordingly, the present disclosure provides enhanced hybridoma generation methods useful in antibody discovery. In exemplary embodiments, the method of generating hybridomas comprises (a) preparing an enriched population of IgG-positive (IgG+) memory B cells from cells obtained from secondary lymphoid organs of one or more immunized non-human animals, wherein the enriched population is substantially devoid of IgM+ B-cells, (b) bulk-culturing at large-scale the enriched population to obtain an expanded population, and (c) fusing cells of the expanded population with myeloma cells to obtain hybridomas. In exemplary aspects, (i) less than or about 10% of the enriched population are IgM-positive (IgM+) B cells and/or (ii) the ratio of the IgG+ memory B cell count to IgM+ B cell count of the enriched population is greater than about 0.5, optionally, greater than about 1 or greater than about 2. Without being bound to a particular theory, the hybridomas produced by the presently disclosed methods allow for deep mining of the immune repertoire of an immunized non-human animal without limitations imposed by B cell antibody secretion rate and/or low abundance of rare B cells. Advantageously, the hybridomas produced by the presently disclosed methods more fully represent the immune repertoire of immunized non-human animals. In exemplary aspects, the percentage of the immortalized IgG+ memory B cell repertoire captured by the presently disclosed methods is maximized and, in various instances, is at least about 15% (e.g., at least about 20% or at least about 25%) of the repertoire produced by the immunized animals. The presently disclosed methods further allow for an unlimited supply of hybridoma supernatant that may be screened for antigen-specific antibodies. In exemplary aspects, the presently disclosed methods routinely yield hundreds, if not thousands, of antigen-specific hybridomas producing antigen-specific antibodies. The presently disclosed methods furthermore lead to higher fusion efficiencies, yielding a highly efficient capture of B cell clones in the hybridoma pool.

Accordingly, the present disclosure additionally provides methods of generating hybridomas. In exemplary embodiments, the method comprises (a) preparing an enriched population of IgG+ memory B cells from cells obtained from secondary lymphoid organs of one or more immunized non-human animals, wherein less than about 10%, optionally, less than about 5%, of the cells of the enriched population are IgM+ B cells; (b) bulk-culturing the enriched population to obtain an expanded population; and (c) fusing cells of the expanded population with myeloma cells to obtain hybridomas. In exemplary aspects, less than 2.5% or less than 1% of the cells of the enriched population are IgM+ B cells. In various aspects, the method comprises removing greater than about 90% (e.g., greater than 95%, greater than 98%, greater than 99%) IgM+ cells and/or positively selecting for IgG+ cells to obtain the enriched population. In exemplary aspects, (i) less than or about 10% of the enriched population are IgM-positive (IgM+) B cells and/or (ii) the ratio of the IgG+memory B cell count to IgM+ B cell count of the enriched population is greater than about 0.5, optionally, greater than about 1 or greater than about 2. In exemplary aspects, the IgM+ B cell count of the enriched population is smaller than the IgM+ B cell count and the ratio of the IgG+ memory B cell count to IgM+ B cell count of the enriched population is greater than 1 or greater than 2. In various aspects, the method comprises bulk-culturing the enriched population at a density of about 350 B220-positive B cells per mL to about 700 B220-positive B cells per mL. In exemplary aspects, bulk-culturing the enriched population is initiated with a seeding density of about 350 B220-positive B cells per mL to about 700 B220-positive B cells, optionally, about 600 B220-positive cells per mL to about 650 B220-positive cells per mL. In exemplary instances, the method comprises fusing cells of the expanded population with myeloma cells at a ratio of B cells to myeloma cells within the range of 1:1 to 1:4, e.g., 1:1.0, 1:1.5, 1:2.0, 1:2.5, 1:3.0, 1:3.5, or 1:4.0. In exemplary aspects, all cells of the expanded population (and thus all B-cells of the expanded population) are combined with myeloma cells. For instance, B cells of the enriched population or expanded population are not selected for fusing with myeloma cells based on production of antibodies which bind to an antigen. Also, for example, B cells of the enriched population or expanded population are not assayed for the production of antigen-specific antibodies prior to fusing with myeloma cells. In various instances, the method comprises screening hybridomas for production of antibodies and, optionally, screening sera obtained from the immunized animals for production of antigen-specific antibodies. In exemplary instances, the only screening for production of antigen-specific antibodies which occurs after harvesting lymphoid organs from immunized animals is the screening of hybridomas.

The present disclosure also provides methods of screening for hybridomas expressing antigen-expressing antibodies, comprising generating hybridomas in accordance with the present disclosure, culturing hybridomas in individual wells, and screening the supernatant of each well for antigen-specific antibodies. In various aspects, about 1 to about 10 or about 1 to about 5 different clones of hybridomas are cultured in a single well.

Further provided are methods of producing antigen-specific antibodies, comprising generating hybridomas in accordance with the present disclosure, culturing hybridomas in individual wells, screening the supernatant of each well for antigen-specific antibodies to identify the hybridomas expressing antigen-specific antibodies; and expanding the culture of the identified hybridomas to produce antigen-specific antibodies.

### BRIEF DESCRIPTION OF THE DRAWINGS

FIG. 1A illustrates an exemplary EHG method of the present disclosure and the following text details the EHG method.

FIG. 1B illustrates an exemplary enrichment process which removes RBCs, non-B cells, and IgM+ cells and also positively selects for surface IgG+ cells to obtain an enriched population which may then be used for bulk culturing.

FIG. 1C illustrates an exemplary enrichment process which removes RBCs from the pooled single cell suspension derived from spleens of immunized animals, followed by combining the RBC-depleted SCS with a SCS derived from LNs of immunized animals Non-B cells and IgM+ cells are removed from the combined SCS using a magnet. Surface IgG+ cells are positively selected for using IgG microbeads. Release of surface IgG+ cells from a column leads to an enriched population which may then be used for bulk culturing.

FIG. 2 is a schematic of an exemplary method for enhanced hybridoma generation.

FIG. 3A is a series of FACS analysis plots for B cell markers to evaluate enrichment for IgG+ memory B cells before B-cell culture and fusion.

FIG. 3B is a series of FACS analysis plots for B cell surface markers to evaluate the enrichment process. This enriched population contains the high affinity IgG secreting plasma cell population and is applied to direct B cell discovery technologies for antigen binding secretion assays.

### DETAILED DESCRIPTION

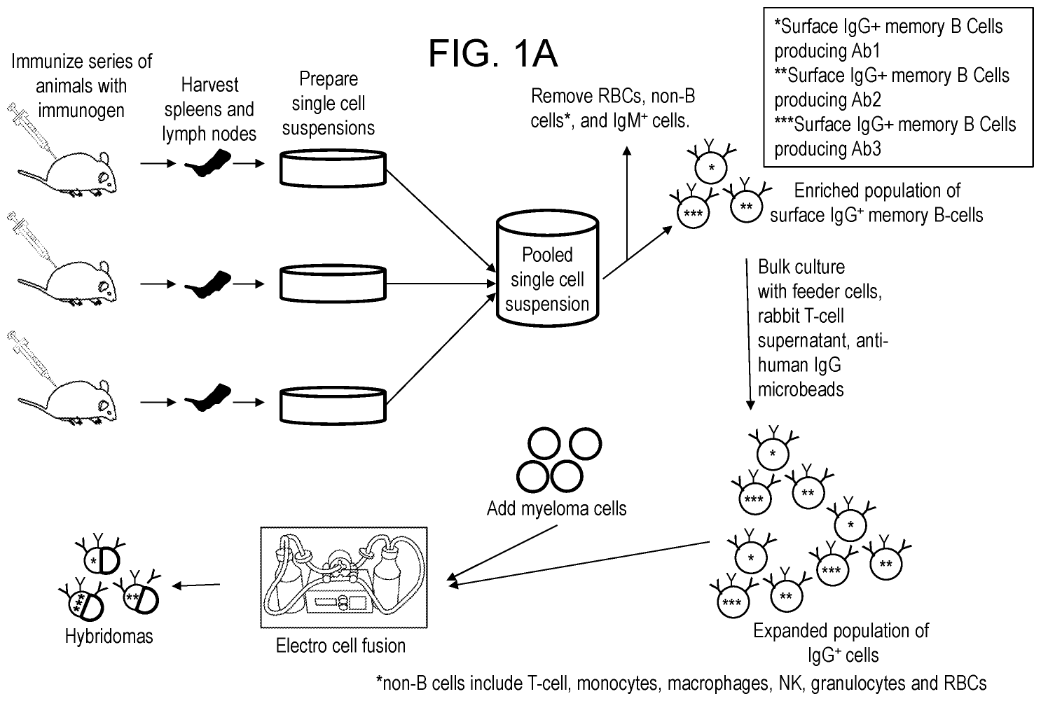

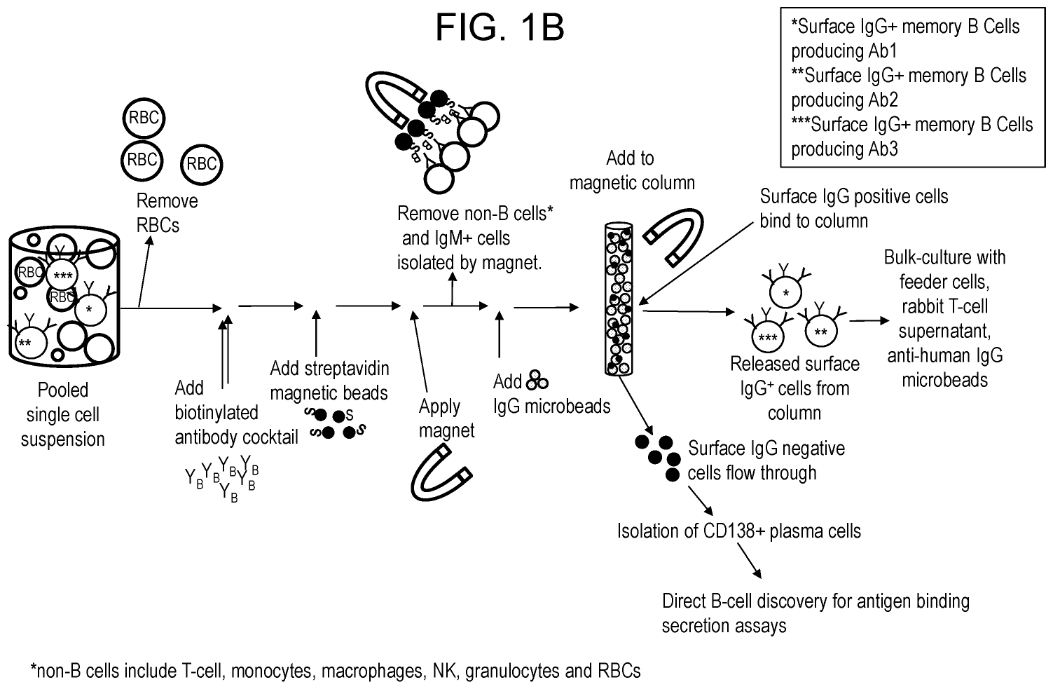

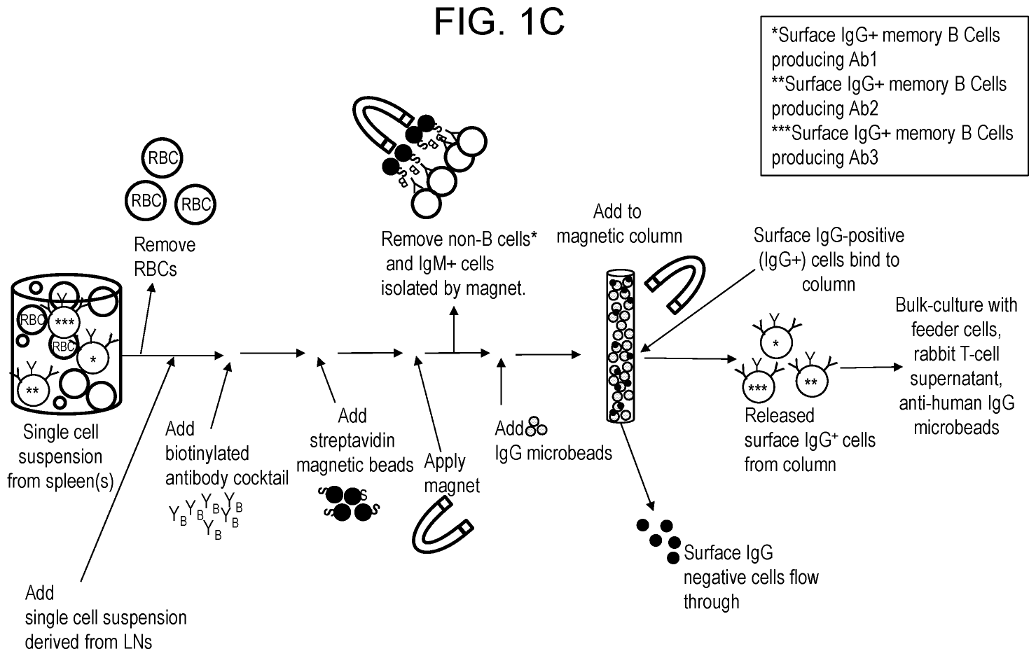

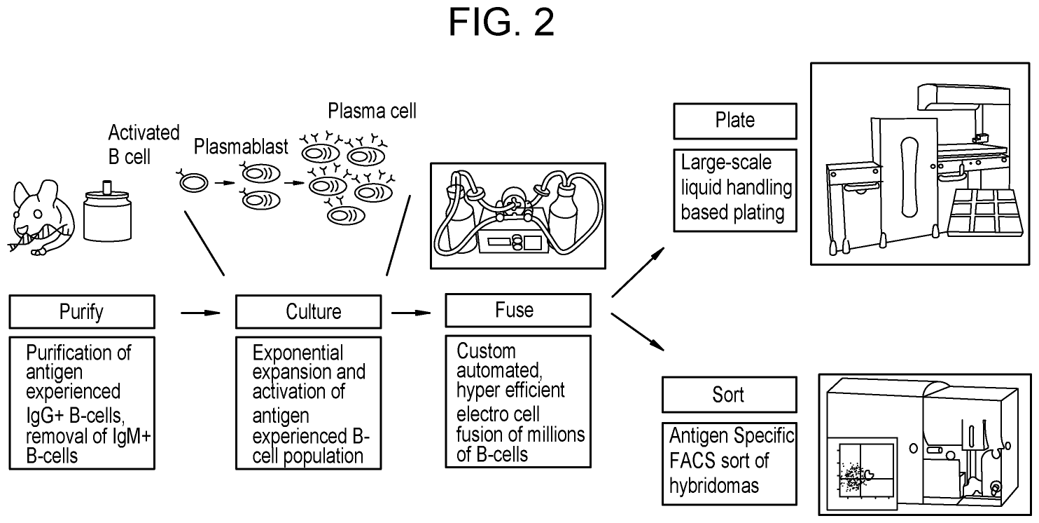

The present disclosure provides enhanced hybridoma generation (EHG) methods which greatly enhance the efficiency of immune repertoire capture in transgenic animals. This is achieved at least in part by expanding highly purified memory B cells in bulk culture for 4-6 days before fusion. In some embodiments, this is achieved at least in part by expanding highly purified memory B cells in bulk culture for 6 days before fusion. In the pre-fusion, bulk culture, each memory B cell clone is estimated to undergo 7-10 divisions (when expanding B cells in bulk culture for 6 days), generating 125-1000 clonal copies. Without being bound by a particular theory, after B cell bulk culture for e.g., 6 days, the high B cell copy number, a blasting phenotype of the activated B cell, the size of the B cell during fusion (approximating the size of the fusion partner, thereby facilitating the fusion), and the fact that B cell membranes are more fluid and thus more amendable to fusion after e.g., 6 days of B cell culture are all factors that overcome the inefficiency of the fusion event. It is estimated that about 25% of the in-vivo memory B cell immune repertoire is captured in the presently disclosed EHG process, thereby providing hybrid pools with deep diversity. In exemplary aspects, the EHG method comprises harvesting immune tissues from immunized transgenic animals and processing cells from such tissues into single cell suspensions (FIG. 1A). In various aspects, non-B cells and IgM-positive B cells are removed to specifically enrich for surface IgG-positive memory B cells and in various instances, about 99.9% of the live cells are removed from the immune tissue preparations while 25-50% of the IgG+ memory B cell population is retained. The highly enriched memory B cell fraction is, in various aspects, bulk cultured in the presence of an irradiated murine T-cell line (EL4B5), rabbit T-cell Supernatant (TSN) and microbeads attached to anti-IgG antibody (e.g., anti-human IgG microbeads). While in culture, the quiescent memory B cells become highly activated and, in some aspects, undergo 7-10 divisions. In various instances, after −6 days of culture, cells are collected and fused with a P3 myeloma cell line to produce hybridomas. The calculation of fusion efficiency is enabled by the measure of clonal outgrowth in low density seeding from a fraction of the hybrid pool. Identification of antigen specific clones starts from here using methods also employed for traditional hybridoma generation.

Without being bound to a particular theory, the EHG method described herein, compared to traditional hybridoma generation, provides a unique ability to capture a much larger fraction of the in-vivo generated immune repertoire in rodents, specifically targeting the IgG+ memory B cell compartment, which is considerably deeper and more diverse than the plasma cell compartment. The EHG methods disclosed herein advantageously overcome limitations of prior methods, including, for instance, poor fusion efficiency and weak immune responses, leading to the production of hybridoma pools with large and diverse antibody repertoires. The EHG methods disclosed herein may be successfully applied to any transgenic mouse and rat models and to wildtype strains. Notably, the EHG methods disclosed herein are also effective in first generation human antibody transgenic animals such as XenoMouse® where the immune response is less robust and antigen-responding B cells are rarer. The presently disclosed EHG methods specifically immortalize the in-vivo generated memory B cell population, while traditional hybridoma generation, as described by Kohler and Milstein, Nature (1975) 256, 495-497, is biased to immortalize the plasma cell population, which represents a different arm of the antigen specific repertoire. It should be noted that even when applying the method of EHG to the IgG+ memory cells, the plasma cell population can be successfully isolated from the same immune tissue before proceeding to EHG and be captured by a variety of single B cell technologies combined with direct molecular rescue. The EHG methods of the present disclosure enable interrogation of both the memory B cell and the plasma cell compartment, adding depth to capture of the in-vivo immune response to antigen.

When considering the outcomes of the traditional vs the EHG method described herein, it must be appreciated that the B cell populations captured during the fusion events are different. The traditional hybridoma generation is strongly biased towards plasma cells and the EHG strongly towards memory cells. This is driven by the enrichment, size and activation state of the B cells. Fusion events are more likely to succeed between cells of equal size, and in cells with a more fluid plasma membrane as found in activated cells (Rems et al., Sci Rep (2013)3, 3382). The fusion partner (e.g., myeloma cell) is close in size to the plasma cell, and so in traditional hybrid generation the rare plasma cells are more likely to contribute to the hybrid pool than the small resting, albeit more numerous memory cells. Therefore, traditional hybridoma generation methods produce relatively small hybrid pools with limited diversity (Dubois et al. Hum Antibodies (2016) June 8; 24(1-2):1-15).

In contrast, in EHG the starting point is the highly enriched IgG+ memory cell compartment, depleted of IgM+ B cells. This depletion is critical as murine IgG+ B cells will undergo 7-9 divisions over 6 days of culture, while IgM+ B cells typically will go through 9-10+ divisions in culture and will also isotype switch to become IgG+ B cells. If cultured together, the IgM+ B cells will dramatically dilute the percent of high affinity IgG derived antigen experienced B cells in the pool. This is the population subsequently fused. The outcomes are hybrid pools with large diversity and a different repertoire than that observed using the traditional method.

The immortalization of a largely uninterrogated source and inclusive pool from a pre-enriched IgG+ memory B cell compartment comprising multiple copies of antigen specific B cells to overcome inherent electro cell fusion inefficiency is the first important distinction setting EHG apart from traditional hybridoma generation. The memory B cell compartment, while not selected in vivo for high affinity binding in first line response to pathogens, can harbour larger diversity and less bias toward dominant antigenic determinants, providing important repertoire not captured from in-vivo generated plasma cells.

The second important distinction is the enabling of antibody discovery from very rare antigen responding B cells derived from animal models such as the transgenic Xenomouse®. Despite low immune cell counts and less robust immune responses in the transgenic platforms, the methods described herein of highly enriching, then activating and expanding the memory cells prior to fusion enables highly efficient capture of immune repertoire and generation of large and diverse hybrid pools, something which is not always possible using traditional hybrid generation in transgenic animals.

Consistent with the foregoing, the present disclosure further provides methods of generating hybridomas, e.g., hybridomas producing antibodies having a desired antigen-specificity. In exemplary embodiments, the method comprises (a) preparing an enriched population of IgG+ memory B cells from cells obtained from secondary lymphoid organs of one or more immunized non-human animals, wherein (i) less than about 10% (e.g., less than about 9%, less than about 8%, less than about 7%, less than about 6%, less than about 5%, less than about 4%, less than about 3%, less than about 2%, less than about 1%) of the cells of the enriched population are IgM-positive (IgM+) B cells and/or (ii) the ratio of the IgG+ memory B cell count to IgM+ B cell count of the enriched population is greater than about 0.5, optionally, greater than about 1 or greater than about 2; (b) bulk-culturing the enriched population to obtain an expanded population; and (c) fusing cells of the expanded population with myeloma cells to obtain hybridomas. In various aspects, the method comprises (a) preparing an enriched population of IgG+ memory B cells from cells obtained from secondary lymphoid organs of one or more immunized non-human animals, wherein less than about 10% (e.g., less than about 9%, less than about 8%, less than about 7%, less than about 6%, less than about 5%, less than about 4%, less than about 3%, less than about 2%, less than about 1%) of the cells of the enriched population are IgM-positive (IgM+) B cells; (b) bulk-culturing the enriched population to obtain an expanded population; and (c) fusing cells of the expanded population with myeloma cells to obtain hybridomas.

Preparing an Enriched Population

In various instances, the method comprises preparing an enriched population of IgG+ memory B cells from secondary lymphoid organs of one or more immunized non-human animal(s) In various aspects, the method comprises preparing an enriched population of IgG+ memory B cells from a single cell suspension dissociated from secondary lymphoid organs of one or more immunized non-human animal(s) In various instances, the secondary lymphoid organs are spleen, lymph nodes Peyer's patches, mucosal tissues (e.g., the nasal associated lymphoid tissues, adenoids, and/or tonsils. Optionally, the secondary lymphoid organs are lymph nodes (LN) (e.g., draining LNs) and/or spleen. In various aspects, the secondary lymphoid organs are or have been harvested from the immunized non-human animal(s) about 3 to about 5 (e.g., about 3, about 4, or about 5) days post-immunization Optionally, the secondary lymphoid organs were harvested from at least 1 to about 15 (e.g., about 1 to about 10) immunized non-human animal(s) about 3 to about 5 (e.g., about 3, about 4, or about 5) days post-immunization. In exemplary aspects, the secondary lymphoid organs are the secondary lymphoid organs harvested from only select immunized non-human animals, optionally, wherein the select immunized non-human animals were selected based on post-immunization serum antibody titer level. For example, in exemplary aspects, the method comprises immunizing animals, wherein only some of the immunized animals are chosen for secondary lymphoid organ harvesting. Optionally, the chosen animals are those that exhibit a level of post-immunization serum antibody titer level that is at or above a threshold level. In various instances, the method comprises immunizing animals and assaying post immunization the sera of all immunized animals, and selecting only a fraction of the immunized animals for secondary lymphoid organ harvest, wherein the selection is based on the post-immunization serum antibody titer levels. In various instances, the post-immunization serum antibody titer levels are post-immunization serum titer levels of antigen-specific antibodies. In exemplary aspects, the method comprises harvesting the secondary lymphoid organs from the immunized non-human animal(s) about 3 to about 5 (e.g., about 3, about 4, or about 5) days post-immunization. In various instances, the method comprises harvesting the secondary lymphoid organs from at least 1, 2, 3, 4, or 5 immunized non-human animals about 3 to about 5 (e.g., about 3, about 4, or about 5) days post-immunization. The immunized non-human animals may be any of those known in the art, including, but not limited to, any of the non-human animals described herein. In various aspects, the non-human animals are mice (e.g., XenoMouse®). Optionally, the non-human animals are rats, e.g., transgenic rats (e.g., UniRat®) or wild-type rats. In various aspects, the immunized non-human animals are or have been immunized according to any protocol known in the art. In various aspects, the non-human animal(s) are or have been immunized according to any of the immunization protocols described herein. See, e.g., Immunization below. In various aspects, the method further comprises (a) immunizing one or more non-human animal(s) with an immunogen, (b) harvesting secondary lymphoid organs from the immunized non-human animal(s), optionally, about 3 to about 5 days post-immunization, (c) preparing a single-cell suspension (SCS) from the secondary lymphoid organs harvested from each immunized non-human animal, and/or (d) preparing a pooled SCS by combining the SCS from the secondary lymphoid organs of more than one immunized non-human animals.

In exemplary aspects, the method comprises preparing a single cell suspension (SCS) from the secondary lymphoid organs of the immunized non-human animals, optionally, the secondary lymphoid organs of select immunized non-human animals. In exemplary instances, the method comprises preparing a single cell suspension comprising mixed immune cells obtained from the secondary lymphoid organs. The single cell suspension may be prepared by dissociating organs on a slide or by using a homogenizer (e.g., Dounce tissue grinder, disperser, microbead homogenizer, ultrasonic processor, blender) and/or a tissue dissociator, e.g., gentleMACS™ (Miltenyi Biotec, Bergisch Gladbach, Germany) with or without a tissue dissociation kit (e.g., MACS® Tissue Dissociation Kit (Miltenyi Biotec), according to methods known in the art. See, e.g., Reichard and Asosingh, Cytometry 95(2): 219-226 (2019), Scheuermann et al., Current Directions in Biomedical Engineering 5(1): 545-548 (2019). In exemplary aspets, the method comprises preparing a SCS from the spleens of one or more immunized non-human animals and/or preparing a SCS from the draining lymph nodes from one or more immunized non-human animals Optionally, two separate SCSs are prepared: one from the pooled spleens of the immunized non-human animals and one from the pooled LNs of the immunized animals. In various instances, the SCS from the pooled spleens is subject to a RBC depletion step followed by combining with the SCS from the pooled LNs to produce a pooled or bulk single cell suspension. Optionally, the RBCs are removed by using an RBC lysing buffer, such as BD Pharm Lyse™ (BD Biosciences, Franklin Lakes, NJ) or Red Blood Cell Lysing Buffer Hybri-Max™ (Millipore Sigma, St. Louis, MO).

In various aspects, the method comprises one or more of (i) immunizing non-human animals with an immunogen, (ii) harvesting secondary lymphoid organs from immunized non-human animals, optionally, about 3 to about 5 days after the last boost with immunogen, (iii) preparing a single cell suspension from secondary lymphoid organs, optionally, by dissociating organs on a slide or by using a homogenizer and/or a tissue dissociator with or without a tissue dissociation kit, and (iv) combining multiple prepared single cell suspensions (e.g., from different immunized non-human animals) to produce a pooled or bulk single cell suspension. In various aspects, an enriched population of IgG+ memory B cells is prepared from the single cell suspension (e.g., pooled or bulk single cell suspension) by removing red blood cells (RBCs), non-B cells and/or IgM-positive (IgM+) cells. In various aspects, the RBCs are removed from the single cell suspension (e.g., pooled or bulk single cell suspension) by using an RBC lysing buffer, such as BD Pharm Lyse™ (BD Biosciences, Franklin Lakes, NJ) or Red Blood Cell Lysing Buffer Hybri-Max™ (Millipore Sigma, St. Louis, MO). In various instances, non-B-cells are removed from the single cell suspension (e.g., pooled or bulk single cell suspension) by using one or more antibodies (e.g., an antibody cocktail) that specifically bind to a cell surface marker of non-B cells. In exemplary aspects, the non-B-cells are one or more of T-cells, monocytes, macrophages, natural killer (NK) cells, granulocytes and RBCs). In exemplary instances, the one or more antibodies are linked to biotin. In various aspects, the method comprises removing T cells, monocytes, macrophages, NK cells, RBCs, granulocytes, or a combination thereof, from the single cell suspension. In exemplary instances, the method comprises removing T cells, monocytes, macrophages, NK cells, RBCs, granulocytes, or a combination thereof, from the single cell suspension using biotin-labeled antibody and streptavidin-labeled beads, optionally, streptavidin-labeled magnetic beads. In various aspects, the cell surface marker of non-B cells is NK1.1 (expressed by NK cells), CD90.2 (expressed by T-cells), Ly-6G GR.1 (expressed by granulocytes and/or macrophages), CD3ε (expressed by T-cells), CD4 (expressed by T cells), CD8a (expressed by T-cells), CD11b (expressed by granulocytes, macrophages, dendritic cells, and/or NK cells) and TER119 (expressed by erythroid cells).

In exemplary aspects, IgM+ cells are removed from the single cell suspension (e.g., pooled or bulk single cell suspension) by adding a biotinylated anti-IgM antibody (e.g., anti-human IgM antibody). In various aspects, the biotinylated antibodies are added to the single cell suspension to allow for the antibodies to bind to the cell surface markers on the non-B cells and/or IgM+ cells. Afterward, in various instances, magnetic beads linked to streptavidin (e.g., streptavidin magnetic beads) are added to allow for the streptavidin to bind to the biotin of the biotinylated antibodies. In exemplary aspects, a magnet is used to isolate and remove the magnetic beads, which are linked to the non-B cells and/or IgM+ cells through the antibodies. In various instances, the method comprises harvesting spleens from immunized non-human animals and preparing a SCS from the spleens, harvesting LNs from the immunized non-human animals and preparing a SCS from the LNs, removing RBCs from the SCS from the spleens, combining the SCS from the LNs and the RBC-depleted spleen-derived SCS to obtain a pooled SCS, removing IgM+ cells from the pooled SCS by adding a biotinylated anti-IgM antibody and capturing IgM+ cells with streptavidin magnetic beads.

Optionally, greater than 90% IgM+ B cells are removed from the pooled SCS to obtain an enriched population. In various instances, greater than 95% (e.g., greater than 96%, greater than 97%, greater than 98%, or greater than 99%) of IgM+ B cells of the pooled SCS are removed to obtain an enriched population substantially depleted of IgM+ cells. In various instances, the method comprises removing IgM+ cells and/or selecting for IgG+ cells. In various aspects, the method comprises adding anti-IgG antibody-labeled magnetic beads (e.g., anti-human IgG antibody-labeled magnetic beads) to an enriched population substantially depleted of IgM+ cells in order to isolate memory B cells. In various aspects, the remaining fraction (e.g., depleted of RBCs, non-B cells and/or IgM+ cells) is incubated with microbeads linked to anti-IgG antibodies, e.g., anti-human IgG antibodies. In certain aspects, through the anti-IgG antibodies, cells expressing IgG on the cell surface bind to the microbeads. In exemplary aspects, the microbead-antibody-cell mixture is added to a magnetic column which retains the microbeads bound to cells expressing surface IgG. The flow-through fraction in various aspects comprises cells negative for expressing surface IgG. In exemplary instances, the cells expressing surface IgG are released from the column to yield an enriched population of IgG+ memory B cells. In various instances, the percentage of the IgG+ cells increases as RBCs, non-B-cells and/or IgM cells are removed from the single cell suspension (e.g., pooled or bulk single cell suspension). In various aspects, the enriched population of IgG+ memory B cells comprises a higher percentage of IgG+ cells compared to that of the single cell suspension. For instance, in various aspects, the percentage of IgG+ cells (relative to the total live cell count) is increased at least 5-fold or 10-fold or more relative to the percentage of IgG+ cells (relative to the total live cell count) of the single cell suspension prior to enrichment. In various aspects, less than 1% of the single cell suspension (prior to enrichment) are IgG+ cells and the percentage of IgG+ cells (relative to the total live cell count) increases to about 5%, about 10%, about 15%, or more after enrichment. In various instances, greater than 5%, greater than 10%, or greater than 15% of the live cells of the enriched population are IgG+ cells and/or greater than 20% cells, greater than 30%, greater than 40%, or greater than 50% of the enriched population are positive for B220 expression. B220 is a B-cell marker. In various instances, less than 10% of cells of the enriched population of IgG+ cells are IgM+ cells. In various aspects, less than 5% of cells of the enriched population of IgG+ cells are IgM+ cells. Optionally, less than about 4%, less than about 3%, less than about 2%, less than about 1%) of the cells of the enriched population are IgM+ cells. In various instances, the ratio of the IgG+ cells to IgM+ cells of the enriched population is increased at least 100-fold, at least 200-fold, at least 300-fold, at least 400-fold, at least 500-fold, at least 600-fold, or more, relative to the ratio of the IgG+ cells to IgM+ cells of the single cell suspension or prior to enrichment. Without being bound to a particular theory, the decreased percentage of IgM+ cells together with the increased percentage of IgG+ cells of the enriched population of IgG+ cells advantageously allow for IgG+ cells to expand in bulk culture to yield an expanded population which may then be fused to myeloma cells to produce hybridomas.

In exemplary aspects, the enriched population is prepared by removing IgM+ cells and/or selecting for IgG+ cells. In various instances, the enriched population is prepared by a negative selection of IgM+ cells and/or a positive selection for IgG+ cells. Optionally, greater than 90% IgM+ B cells are removed upon the negative selection and/or the positive selection, and in some instances, greater than 95% IgM+ B cells are removed upon the negative selection and/or the positive selection. In various aspects, greater than 98% IgM+ B cells are removed upon the negative selection, and greater than 99% IgM+ B cells are removed upon the negative selection and the positive selection. Optionally, greater than 99% of the IgM+ cells present in the single cell suspension are removed through the negative selection and the positive selection. In various instances, the ratio of the IgG+ memory B cell count to IgM+ B cell count increases by at least about 50-fold, at least about 60-fold, at least about 70-fold, at least about 80-fold, at least about 90-fold, or at least about 100-fold, upon the negative selection and the positive selection.

Bulk-Culturing

In exemplary aspects of the presently disclosed EHG method, the enriched population is bulk-cultured with anti-IgG antibody-labeled beads (e.g., anti-human IgG antibody-labeled beads) and feeder cells in a cell culture medium comprising rabbit T-cell supernatant By “bulk-culture” is meant that the cell culture is not a clonal cell population. The “bulk culture” in various aspects is a mixture of cells originating from the secondary lymphoid organs harvested from multiple immunized non-human animals. In various instances, “bulk culturing” as used herein refers to culturing a polyclonal mixture of surface IgG-positive B-cells under conditions that will activate them and induce proliferation and differentiation. The feeder cells express CD40L and/or are gamma-irradiated in some instances. Optionally, the feeder cells are gamma-irradiated, CD40L-positive EL4B5 feeder cells. In various aspects, the myeloma cells are in a log phase growth stage. In certain aspects, the myeloma cells are P3 myeloma cells. In exemplary aspects, the enriched population of IgG+ cells is bulk-cultured at a density of 350 B220-positive cells per mL to about 700 B220-positive cells per mL. Optionally, the enriched population of IgG+ cells is bulk-cultured at a density of about 350 B220-positive cells per mL to about 650 B220-positive cells per mL, about 350 B220-positive cells per mL to about 600 B220-positive cells per mL, about 350 B220-positive cells per mL to about 550 B220-positive cells per mL, about 350 B220-positive cells per mL to about 500 B220-positive cells per mL, about 350 B220-positive cells per mL to about 450 B220-positive cells per mL, about 350 B220-positive cells per mL to about 400 B220-positive cells per mL, about 400 B220-positive cells per mL to about 700 B220-positive cells per mL, about 450 B220-positive cells per mL to about 700 B220-positive cells per mL, about 500 B220-positive cells per mL to about 700 B220-positive cells per mL, about 550 B220-positive cells per mL to about 700 B220-positive cells per mL, about 600 B220-positive cells per mL to about 700 B220-positive cells per mL, about 650 B220-positive cells per mL to about 700 B220-positive cells per mL. Optionally, the enriched population of IgG+ cells is bulk-cultured at a density of about 550 B220-positive cells per mL to about 650 B220-positive cells per mL, optionally, about 625 B220-positive cells per mL. In various instances, bulk-culturing the enriched population is initiated with a seeding density of about 350 B220-positive B cells per mL to about 700 B220-positive B cells, optionally, about 600 B220-positive cells per mL to about 650 B220-positive cells per mL. In various aspects, the seeding density is about 350 B220-positive cells per mL to about 650 B220-positive cells per mL, about 350 B220-positive cells per mL to about 600 B220-positive cells per mL, about 350 B220-positive cells per mL to about 550 B220-positive cells per mL, about 350 B220-positive cells per mL to about 500 B220-positive cells per mL, about 350 B220-positive cells per mL to about 450 B220-positive cells per mL, about 350 B220-positive cells per mL to about 400 B220-positive cells per mL, about 400 B220-positive cells per mL to about 700 B220-positive cells per mL, about 450 B220-positive cells per mL to about 700 B220-positive cells per mL, about 500 B220-positive cells per mL to about 700 B220-positive cells per mL, about 550 B220-positive cells per mL to about 700 B220-positive cells per mL, about 600 B220-positive cells per mL to about 700 B220-positive cells per mL, or about 650 B220-positive cells per mL to about 700 B220-positive cells per mL. Optionally, the seeding density is about 550 B220-positive cells per mL to about 650 B220-positive cells per mL, optionally, about 625 B220-positive cells per mL. In various aspects, the volume of the bulk culture is about 10 mL or more, about 20 mL or more, about 30 mL or more, about 40 mL or more, about 50 mL or more. In various instances, the volume of the bulk culture is greater than 50 mL, greater than 60 mL, greater than 70 mL, greater than 80 mL, or greater than 90 mL. In various aspects, the volume is greater than 100 mL, greater than 250 mL, greater than 500 mL, greater than 750 mL, about 1.0 L, about 1.1 L, about 1.2 L, about 1.3 L, about 1.4 L, about 1.5 L, about 1.6 L, about 1.7 L, about 1.8 L, about 1.9 L, or about 2.0 L. The enriched population is bulk-cultured in a volume of about 50 mL to about 500 mL, optionally, about 100 mL to about 300 mL in exemplary instances. Optionally, the enriched population is bulk-cultured for at least about 4 days, at least about 5 days, or at least about 6 days. In exemplary aspects, the enriched population of IgG+ cells is bulk-cultured for at least about 4 days. In exemplary aspects, the enriched population of IgG+ cells is bulk-cultured for at least about 5 days. In exemplary instances, the enriched population of IgG+ cells is bulk-cultured for about 6 days. In various instances, the cells of the enriched population undergo at least or about 5 cell divisions to about 12 cell divisions. In various instances, the cells of the enriched population undergo at least or about 6 cell divisions to about 12 cell divisions (e.g., at least or about 6 cell divisions to about 11 cell divisions, at least or about 6 cell divisions to about 10 cell divisions), optionally, at least or about 7 cell divisions to about 10 cell divisions, e.g., about 7, about 8, about 9, or about 10 cell divisions, to yield the expanded population.

Cell Fusion

In exemplary aspects, the cells of the expanded population are fused with myeloma cells by electro cell fusion (ECF) to obtain hybridomas, optionally, wherein the ECF is carried out using an SDF Fusion Chamber. In various instances, all cells (including all B cells) of the expanded population are used for fusing with myeloma cells. In various aspects, all cells (including all B cells) of the expanded population are combined with myeloma cells for fusing. In exemplary instances, the method does not comprise selecting B cells for fusing with myeloma cells. In exemplary instances, the method does not comprise selecting B cells based on production of antibodies which bind to an antigen (e.g., antigen-specific antibodies) for fusing with myeloma cells. In exemplary aspects, B cells of the enriched population or the expanded population are not screened for production of antibodies which bind to an antigen (e.g., antigen-specific antibodies).

In various instances, the B-cells are present during the ECF at a B-cell to myeloma cell ratio of about 1:1 to about 1:4 (e.g., 1:1.5, 1:2.0, 1:2.5, 1:3.0, 1:3.5, 1:4.0). In some aspects, the ratio is about 1:2. Methods of electro cell fusion for hybridoma production are described in the art. See, e.g., Greenfield, Cold Spring Harbor Protocols; doi:10.1101/pdb.prot103184 (2019). Optionally, cells of the expanded population are fused with myeloma cells in a volume greater than 10 mL per fusion event. In various aspects, the method further comprises separating or isolating hybridomas from unfused cells. In various instances, the method comprises transferring the cells from the ECF chamber to culture medium comprising hypoxanthine azaserine (HA) which leads to the cell death of any unfused cells. In various aspects, the method comprises transferring the cells from the ECF chamber to culture medium comprising HA for about 3 days or more. The hybridomas in various instances are subsequently stored under frozen conditions. Optionally, after frozen storage, the hybridomas are plated in multi-well plates, or antigen sorted by FACS clonally into multi-well plates. In exemplary aspects, each well comprises up to 5 hybridoma clones per well. In exemplary instances, the supernatant from each well is used in one or more screening assays to detect and characterize the antibodies produced by the hybridomas in the well. In various aspects, the supernatant is assayed for antigen-specific antibodies, optionally, by ELISA, FACS, or other technique.

In various aspects, the method further comprises screening the hybridomas for production of antibodies which bind to an antigen and/or culturing hybridomas in multiplate wells and screening the supernatant of each well for antigen-specific antibodies. Optionally, the screening comprises an immunoassay which detects binding of antibodies to the antigen. The immunoassay is in various aspects a fluorescence activated cell sorting (FACS) analysis. In various aspects, the screening for cells producing antigen-specific antibodies occurs only after hybridomas are obtained, and not before hybridomas are obtained. Optionally, the only time assaying for antigen-specific antibodies occurs after harvest of secondary lymphoid organs is after hybridomas are obtained. In exemplary instances, screening for antigen-specific antibodies occurs only before secondary lymphoid organs are harvested and after hybridomas are obtained. Optionally, the screening for antigen-specific antibodies that occurs before secondary lymphoid organs are harvested comprises a titer analysis of serum obtained from live immunized animals.

In exemplary embodiments, the method of generating hybridomas producing antigen-specific antibodies, comprises:
a immunizing one or more non-human animal(s) with an immunogen;
b. harvesting secondary lymphoid organs from the immunized non-human animal(s);
c. preparing a single-cell suspension (SCS) from the secondary lymphoid organs harvested from each immunized non-human animal;
d. preparing a pooled SCS by combining all SCSs prepared in (c);
e. removing greater than 95% IgM+ cells from the pooled SCS and/or positively selecting for surface IgG+ cells from the pooled SCS to obtain an enriched population of IgG-positive (IgG+) memory B cells, wherein:
   i. less than or about 10% of the enriched population are IgM-positive (IgM+) B cells and/or
   ii. the ratio of the IgG+ memory B cell count to IgM+ B cell count of the enriched population is greater than 0.5, optionally, greater than 1 or greater than 2,
f. bulk-culturing the enriched population to obtain an expanded population;
g. fusing cells of the expanded population with myeloma cells to obtain hybridomas; and
h. identifying the hybridomas producing antigen-specific antibodies by culturing single hybridomas in individual wells and screening the supernatant of each well for antigen-specific antibodies.

In exemplary embodiments of the presently disclosed methods of generating hybridomas producing antigen-specific antibodies, the hybridomas obtained represent at least 15% of the IgG+ memory B cell repertoire produced by the immunized animals. In various instances, the hybridomas obtained represent at least 20% of the IgG+ memory B cell repertoire produced by the immunized animals Optionally, the hybridomas obtained represent at least 25% of the IgG+ memory B cell repertoire produced by the immunized animals.

In exemplary instances of the presently disclosed EHG methods, greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of the total IgG+ memory B cells from the in-vivo repertoire are recovered after enrichment and entered into bulk culture. In other exemplary aspects of the presently disclosed EHG methods, greater than 10% (e.g., greater than 15%, greater than 20%, greater than 25% or more) of the total IgG+ memory B cells from the in-vivo repertoire are recovered after enrichment and entered into bulk culture. In exemplary instances of the presently disclosed EHG methods, greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of the memory B cell repertoire of B-cells pooled from the immunized animals is captured. In other exemplary instances of the presently disclosed EHG methods, greater than 10%, e.g., greater than 15%, of the memory B cell repertoire of B-cells pooled from the immunized animals is captured. In various aspects, greater than 100,000 unique hybridomas are generated by the presently disclosed EHG methods. In certain instances, greater than 150,000 or greater than 200,000 unique hybridomas are generated presently disclosed EHG methods. In various instances, the fusion efficiency achieved by the presently disclosed EHG methods is at least 0.10%. In other instances, the fusion efficiency achieved by the presently disclosed EHG methods is greater than 0.001%, 0.005%, 0.01%, 0.02%, 0.03%, 0.04%, 0.05%, 0.06%, 0.07%, 0.08%, or 0.09%. In other instances, the fusion efficiency achieved by the presently disclosed EHG methods is greater than 0.10%, 0.11%, 0.12%, 0.13%, 0.14%, 0.15%, 0.16%, 0.17%, 0.18%, 0.19%, or 0.20%. In a particular embodiment, the fusion efficiency achieved by the presently disclosed EHG methods is greater than 0.140%.

Methods of screening for hybridomas expressing antigen-specific antibodies are additionally provided herein. In exemplary embodiments, the method comprises (a) generating hybridomas in accordance with any one of the presently disclosed methods of generating hybridomas, (b) culturing hybridomas in wells, optionally, wherein each well comprises up to 5 hybridomas; and (c) screening or assaying the supernatant of each well for antigen-specific antibodies. In exemplary embodiments, the method comprises (a) preparing an enriched population of IgG+ memory B cells from cells obtained from secondary lymphoid organs of one or more immunized non-human animals, wherein less than about 5% of the cells of the enriched population are IgM+ B cells; (b) bulk-culturing the enriched population to obtain an expanded population; (c) fusing cells of the expanded population with myeloma cells to obtain hybridomas; (d) culturing hybridomas in wells; and (e) screening the supernatant of each well for antigen-specific antibodies. The screening in various aspects comprises an ELISA or binding to streptavidin beads coated by the target antigen, FACS detection of antibody binding to cells transfected by the target, or another high throughput microscopic technique.

The present disclosure also provides methods of producing antigen-specific antibodies. In exemplary embodiments, the method comprises (a) preparing an enriched population of IgG+ memory B cells from cells obtained from secondary lymphoid organs of one or more immunized non-human animals, wherein less than about 10%, e.g., less than about 5%, of the cells of the enriched population are IgM+ B cells; (b) bulk-culturing the enriched population to obtain an expanded population; (c) fusing cells of the expanded population with myeloma cells to obtain hybridomas; (d) culturing hybridomas in wells; (e) screening the supernatant of each well for antigen-specific antibodies to identify the hybridomas expressing antigen-specific antibodies; and (f) expanding the hybridomas identified in (e) to produce antigen-specific antibodies.

Immunization

In various aspects of the present disclosure, the method comprises immunizing a non-human animal with an immunogen. As used herein, the term “immunizing” refers to performing or carrying out an “immunization campaign” or “immunization protocol” or “campaign” to mount an immune response against said immunogen. In exemplary aspects, the immune response comprises a B-cell immune response and/or a humoral immune response against said immunogen. Suitable techniques for immunizing the non-human animal are known in the art. See, e.g., Goding, Monoclonal Antibodies: Principles and Practice, 3 rd ed., Academic Press Limited, San Diego, C A, 1996. The gene gun method described in, e.g., Barry et al., Biotechniques. 16(4):616-8, 620 (1994); Tang et al., Nature. 12; 356(6365):152-4 (1992); Bergmann-Leitner and Leitner, Methods Mol Biol 1325: 289-302 (2015); Aravindaram and Yang, Methods Mol Biol 542: 167-178 (2009); Johnston and Tang, Methods Cell Biol 43 PtA: 353-365 (1994); and Dileo et al., Human Gene Ther 14(1): 79-87 (2003), also may be used for immunizing the non-human animal. Furthermore, as exemplified herein, the immunizing may comprise administering cells expressing the antigen to the non-human animal or administering antigen-loaded dendritic cells, tumor cell vaccines, or immune-cell based vaccines. See, e.g., Sabado et al., Cell Res 27(1): 74-95 (2017), Bot et al., “Cancer Vaccines” in Plotkin's Vaccines, 7^{th} ed., Editors: Plotkin et al., Elsevier Inc., 2018, and Lee and Dy, “The Current Status of Immunotherapy in Thoraic Malignancies” in Immune Checkpoint Inhibitors in Cancer, Editors: Ito and Ernstoff, Elsevier Inc., 2019. In various instances, the immunizing may be carried out by microneedle delivery (see, e.g., Song et al., Clin Vaccine Immunol 17(9): 1381-1389 (2010)); with virus-like particles (VLPs) (see, e.g., Temchura et al., Viruses 6(8): 3334-3347 (2014)); or by any means known in the art. See, e.g., Shakya et al., Vaccine 33(33): 4060-4064 (2015) and Cai et al., Vaccine 31(9): 1353-1356 (2013). Additional strategies for immunization and immunogen preparation, including, for example, adding T cell epitopes to antigens, are described in Chen and Murawsky, Front Immunol 9: 460 (2018).

In various aspects, the method comprises immunizing a non-human animal with an immunogen and said immunogen is administered to the non-human animal one or more (e.g., 2, 3, 4, 5, or more) times. In various aspects, the immunogens are administered by injection, e.g., intraperitoneal, subcutaneous, intramuscular, intradermal, or intravenous. In various aspects, the method comprises immunizing a non-human animal by administering a series of injections of the immunogen. In exemplary aspects, each administration, e.g., injection, is given to the non-human animal about 10 days to about 18 days apart, optionally, about 12 to about 16 days apart, or about 14 days apart. In exemplary aspects, each administration, e.g., injection, is given to the non-human animal more frequently than about 10 days to about 18 days apart. For instance, in exemplary aspects, the timing between administration of the immunogen to the non-human animal is about 1 to about 9 days apart, optionally, about 1 day to about 8 days, about 1 day to about 7 days, about 1 day to about 6 days, about 1 day to about 5 days, about 1 day to about 4 days, about 1 day to about 3 days, about 1 day to about 2 days, about 2 days to about 9 days, about 3 days to about 9 days, about 4 days to about 9 days, about 5 days to about 9 days, about 6 days to about 9 days, about 7 days to about 9 days, about 8 days to about 9 days, about 4 to about 8 days, about 4 days to about 8 days, or about 6 days to about 8 days. The timing between administration of the immunogen to the non-human animal is in various aspects longer. For instance, the timing between administration of the immunogen to the non-human animal may be about 1 to about 20 weeks or longer, e.g., about 1 to about 20 months. Optionally, the timing between administration of the immunogen to the non-human animal is about 1 week to about 19 weeks, about 1 week to about 18 weeks, about 1 week to about 17 weeks, about 1 week to about 16 weeks, about 1 week to about 15 weeks, about 1 week to about 14 weeks, about 1 week to about 13 weeks, about 1 week to about 12 weeks, about 1 week to about 11 weeks, about 1 week to about 10 weeks, about 1 week to about 9 weeks, about 1 week to about 8 weeks, about 1 week to about 7 weeks, about 1 week to about 6 weeks, about 1 week to about 5 weeks, about 1 week to about 4 weeks, about 1 week to about 3 weeks, about 1 week to about 2 weeks, about 2 weeks to about 20 weeks, about 3 weeks to about 20 weeks, about 4 weeks to about 20 weeks, about 5 weeks to about 20 weeks, about 6 weeks to about 20 weeks, about 7 weeks to about 20 weeks, about 8 weeks to about 20 weeks, about 9 weeks to about 20 weeks, about 10 weeks to about 20 weeks, about 11 weeks to about 20 weeks, about 12 weeks to about 20 weeks, about 13 weeks to about 20 weeks, about 14 weeks to about 20 weeks, about 15 weeks to about 20 weeks, about 16 weeks to about 20 weeks, about 17 weeks to about 20 weeks, about 18 weeks to about 20 weeks, or about 19 weeks to about 20 weeks. Optionally, about 1 week to about 8 days, about 1 day to about 7 days, about 1 day to about 6 days, about 1 day to about 5 days, about 1 day to about 4 days, about 1 day to about 3 days, about 1 day to about 2 days, about 2 days to about 9 days, about 3 days to about 9 days, about 4 days to about 9 days, about 5 days to about 9 days, about 6 days to about 9 days, about 7 days to about 9 days, about 8 days to about 9 days, about 4 to about 8 days, about 4 days to about 8 days, or about 6 days to about 8 days. In various instances, during the immunization, each administration (e.g., injection) of immunogen is carried out with the same (A) immunogen, adjuvant, immunomodulatory agent, or combination thereof, (B) amount or dose of immunogen, adjuvant, immunomodulatory agent, or combination thereof, (C) administration route or method of delivering the immunogen, (D) administration site on the non-human animal, or (E) a combination thereof. Alternatively, one or more administrations (e.g., injections) of immunogen during the immunization is performed with a different (A) immunogen, adjuvant, immunomodulatory agent, or combination thereof, (B) amount or dose of immunogen, adjuvant, immunomodulatory agent, or combination thereof, (C) administration route or method of delivering the immunogen, (D) administration site on the non-human animal, or (E) a combination thereof. Optionally, the amount of immunogen decreases or increases with subsequent administrations, e.g., injections. In some aspects, every other administration, e.g., injection, comprises a decreased or increased amount of immunogen, relative to the first and third injections. Exemplary immunizations are described in the examples provided herein.

Non-Human Animals

Advantageously, the presently disclosed methods are not limited to any particular non-human animal. The non-human animal in exemplary aspects, is any non-human mammal. In exemplary aspects, the non-human animal is a mammal, including, but not limited to, mammals of the order Rodentia, such as mice, rats, guinea pigs, gerbils and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the non-human mammal is of the order Primates, Ceboids, or Simoids (monkeys) or of the order Anthropoids (apes). In various aspects, the non-human animal is a goat, llama, alpaca, chicken, duck, fish (e.g., salmon), sheep, or ram.

In exemplary instances, the non-human animal(s) used in the presently disclosed methods are modified, e.g., genetically modified, such that they produce chimeric or fully human antibodies. Such non-human animals are referred to as transgenic animals. The production of human antibodies in transgenic animals is described in Bruggemann et al., Arch Immunol Ther Exp (Warsz) 63(2): 101-108 (2015). Any transgenic animal can be use in the present invention including, but not limited to, transgenic chickens (e.g., OmniChicken®), transgenic rats (e.g., OmniRat®), transgenic llamas, and transgenic cows (e.g., Tc Bovine™). In a particular embodiment, the non-human animal is transgenic mouse such as XenoMouse®, Alloy mouse, Trianni mouse, OmniMouse®, and HuMAb-Mouse®. XenoMouse® is a strain of transgenic mice that produce full-human antibodies. An overview of XenoMouse® is provided by Foltz et al., Immunol Rev 270(1): 51-64 (2016) and U.S. Pat. No. 5,939,598. In exemplary aspects, the non-human animal is a transgenic rat. The transgenic rat in various aspects is Unirat® or OmniFlic®, which is described in Clarke et al., Front Immunol 9:3037 (2019); doi: 10.3389/fimmu.2018.03037 and Harris et al., Front Immunol 9:889 (2018): doi: 10.3389/fimmu.2018.00889, respectively.

Immunogens

Advantageously, the presently disclosed methods are not limited to any particular immunogen. The immunogen in various aspects may be any antigen, optionally, a protein, or a fragment, fusion, or variant thereof. In various instances, the immunogen is a cytokine, lymphokine, hormone, growth factor, extracellular matrix protein, tumor associated antigen, tumor associated antigen, checkpoint inhibitor molecule, cell surface receptor, or a ligand thereof. For purposes of merely illustrating exemplary immunogens, the immunogen used in immunizing the non-human animal may be the target or antigen to which any one of the following antibodies bind: Muromonab-CD3 (product marketed with the brand name Orthoclone Okt30), Abciximab (product marketed with the brand name Reopro®), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-1H®), Adalimumab (product marketed with the brand name Humira®), Tositumomab-I131 (product marketed with the bmnd name Bexxar®), Efalizumab (product marketed with the brand name Raptiva®), Cetuximab (product marketed with the brand name Erbitux®), Ibritumomab tiuxetan (product marketed with the brand name Zevalin®), Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), Eculizumab (product marketed with the bmnd name Soliris®), Certolizumab pegol (product marketed with the brand name Cimzia®), Golimumab (product marketed with the brand name Simponi®), Canakinumab (product marketed with the brand name Ilaris0), Catumaxomab (product marketed with the bmnd name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the bmnd name Prolia®), Belimumab (product marketed with the brand name Benlysta®), Raxibacumab, Ipilimumab (product marketed with the brand name Yervoy0), and Pertuzumab (product marketed with the brand name Perjeta®). In exemplary embodiments, the antibody is one of anti-TNF alpha antibodies such as adalimumab, infliximab, etanercept, golimumab, and certolizumab pegol; anti-IL1β antibodies such as canakinumab; anti-IL12/23 (p40) antibodies such as ustekinumab and briakinumab; and anti-IL2R antibodies, such as daclizumab.

Methods of preparing an immunogen for use in the immunization step are known in the art. See, e.g., Fuller et al., Curr Protoc Mol Biol, Chapter 11, Unit 11.4, (2001); Monoclonal Antibodies: Methods and Protocols, 2^{nd} ed., Ossipow et al. (Eds.), Humana Press 2014. In various instances, the immunogen is mixed with an adjuvant or other solution prior to administration to the non-human animal Many adjuvants are known in the art, and include, in exemplary instances, comprises an oil, an alum, aluminum salt, or a lipopolysaccharide. In various aspects, the adjuvant is inorganic. In alternative aspects, the adjuvant is organic. In various aspects, the adjuvant comprises: alum, aluminum salt (e.g., aluminum phosphate, aluminum hydroxide), Freund's complete adjuvant, Freund's incomplete adjuvant, RIBI adjuvant system (RAS), Lipid A, Sigma Adjuvant System®, TiterMax® Classic, TiterMax® Gold, a Montanide vaccine adjuvant (e.g., Montanide 103, Montanide ISA 720, Montanide incomplete Seppic adjuvant, Montanide ISA51), AF03 adjuvant, AS03 adjuvant, Specol, SPT, nanoemulsion, VSA3, oil or lipid-based solution, (e.g., squalene, MF59®, QS21, saponin, monophosphoryl lipid A (MPL)), trehalose dicorynomycolate (TDM), sTDM adjuvant, virosome, and PRR Ligands See, e.g., “Vaccine Adjuvants Review” at https://www.invivogen.com/review-vaccine-adjuvants and “Role of Adjuvants in Antibody Production”, The Protein Man's Blog: A Discussion of Protein Research, posted on Jun. 2, 2016, at https://info.gbiosciences.com/blog/role-of-adjuvants-in-antibody-production. In various instances, the adjuvant comprises a surface-active substance such as lysolecithin, pluronic polyols, polyanions, peptides, oil emulsions, keyhole limpet hemocyanin, and dinitrophenol. BCG (bacilli Calmette-Guerin) and Corynebacterium parvum.

Antibodies

Although antibody structures vary between species, as used herein, the term “antibody” generally refers to a protein having a conventional immunoglobulin format, typically comprising heavy and light chains, and comprising variable and constant regions. Antibodies obtained or isolated by the present method can have a variety of uses. For example, antibodies obtained by the present method can be used as therapeutics. The antibodies obtained by the present method can also be used as non-therapeutic antibodies as, for example, reagents used in diagnostic assays, e.g., diagnostic imaging assays, and for other in vitro or in vivo immunoassays, e.g., Western blots, radioimmunassays, ELISA, EliSpot assay, and the like. In various aspects, the antibody can be a monoclonal antibody or a polyclonal antibody. In exemplary instances, the antibody is a mammalian antibody, e.g., a mouse antibody, rat antibody, rabbit antibody, goat antibody, horse antibody, chicken antibody, hamster antibody, pig antibody, human antibody, alpaca antibody, camel antibody, llama antibody, and the like. In some aspects, the antibody can be a monoclonal antibody or a polyclonal antibodies optionally produced by a transgenic animal. In such embodiments, the antibodies produced are chimeric antibodies comprising sequences of two or more species. In various instances, an antibody has a human IgG which is a “Y-shaped” structure of two identical pairs of polypeptide chains, each pair having one “light” (typically having a molecular weight of about 25 kDa) and one “heavy” chain (typically having a molecular weight of about 50-70 kDa). A human antibody has a variable region and a constant region. In human IgG formats, the variable region is generally about 100-110 or more amino acids, comprises three complementarity determining regions (CDRs), is primarily responsible for antigen recognition, and substantially varies among other antibodies that bind to different antigens. See, e.g., Janeway et al., “Structure of the Antibody Molecule and the Immunoglobulin Genes”, Immunobiology: The Immune System in Health and Disease, 4^{th} ed. Elsevier Science Ltd./Garland Publishing, (1999). Briefly, in a human antibody scaffold, the CDRs are embedded within a framework in the heavy and light chain variable region where they constitute the regions largely responsible for antigen binding and recognition. A human antibody variable region comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra). Human light chains are classified as kappa and lambda light chains Human heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the disclosure include all such classes or isotypes of human antibodies. The human light chain constant region can be, for example, a kappa- or lambda-type light chain constant region. The heavy chain constant region can be, for example, an alpha-, delta-, epsilon-, gamma-, or mu-type heavy chain constant regions. Accordingly, in exemplary embodiments, the antibody is an antibody of isotype IgA, IgD, IgE, IgG, or IgM, including any one of IgG1, IgG2, IgG3 or IgG4.

Antigen-binding proteins may have structures varying from that of a human antibody. In exemplary instances, the antigen-binding protein comprises only heavy chain fragments, e.g., heavy chain variable region, heavy chain constant region CH2, heavy chain constant region CH3. In various instances, the antigen-binding protein comprises a structure of a small antibody or a nanobody, such as those made by dromedary camel, llama, and shark. See, e.g., Leslie, Science, “Mini-antibodies discovered in sharks and camels could lead to drugs for cancer and other diseases”, 2018, at https://www.sciencemag.org/news/2018/05/mini-antibodie s-discovered-sharks-and-camels-could-lead-drugs-cancer-and-other-diseases.

### EXEMPLARY EMBODIMENTS

The following describe exemplary embodiments of the present disclosure.
1. A method of generating hybridomas, comprising
   a. preparing an enriched population of IgG-positive (IgG+) memory B cells from cells obtained from secondary lymphoid organs of one or more immunized non-human animals, wherein less than about 5% of the cells of the enriched population are IgM-positive (IgM+) B cells;
   b. bulk-culturing the enriched population to obtain an expanded IgG+ memory B cell population; and
   c. fusing cells of the expanded IgG+ memory B cell population with myeloma cells to obtain hybridomas.
2. The method of embodiment 1, wherein the secondary lymphoid organs are secondary lymphoid organs harvested from (i) the immunized non-human animal(s) about 3 to about 5 days post-immunization and/or (ii) at least 1, 2, 3, 4, or 5 immunized non-human animal(s)
3. The method of embodiment 1 or 2, comprising preparing an enriched population of IgG+ memory B cells from a single-cell suspension prepared from the secondary lymphoid organs.
4. The method of any one of embodiments 1-3, wherein the secondary lymphoid organs are spleen and/or lymph nodes.
5. The method of any one of embodiments 1-4, wherein the non-human animals are mice or rats.
6. The method of any one of embodiments 1-5, comprising removing non-B cells, red blood cells (RBCs), IgM-positive (IgM+) cells, or a combination thereof, from a single cell suspension prepared from the secondary lymphoid organs, optionally, comprising removing non-B cells, RBCs, IgM+ cells, or a combination thereof, using antibodies specific to one or more cell surface markers expressed by the non-B cells, RBCs, or IgM+ cells.
7. The method of embodiment 6, wherein the cell surface markers are human IgM, CD90.2, Ly-6G GR.1, NK-1.1, CD3epsilon, CD4, CD8a, CD1 lb, and/or TER119.
8. The method of embodiment 6 or 7, wherein the antibodies are linked to biotin and the method comprises using streptavidin-labeled beads, optionally, streptavidin-labeled magnetic beads, to remove the non-B cells, RBCs, and/or IgM+ cells.
9. The method of any one of embodiments 1-8, comprising selecting for surface IgG+ cells, optionally, by using anti-IgG antibody-labeled beads, optionally, anti-human IgG antibody-labeled magnetic beads.
10. The method of any one of embodiments 1-9, wherein at least 10% cells of the enriched population are IgG+ B cells and/or greater than 20% cells of the enriched population are positive for B220 expression.
11. The method of any one of embodiments 1-10, comprising bulk-culturing the enriched population of IgG+ cells with anti-human IgG antibody-labeled beads and feeder cells in a cell culture medium comprising rabbit T-cell supernatant.
12. The method of embodiment 11, wherein the feeder cells express CD40L and/or are gamma-irradiated.
13. The method of embodiment 12, wherein the feeder cells are gamma-irradiated, CD40L-positive EL4B5 feeder cells.
14. The method of any one of embodiments 1-13, wherein the myeloma cells are in a log phase growth stage.
15. The method of any one of embodiments 1-14, wherein the myeloma cells are P3 myeloma cells.
16. The method of any one of embodiments 1-15, wherein the enriched population is bulk-cultured at a density of about 350 B220-positive B cells per mL to about 700 B220-positive B cells, optionally, about 600 B220-positive cells per mL to about 650 B220-positive cells per mL.
17. The method of anyone of embodiments 1-16, wherein the enriched population is bulk-cultured in a volume of about 50 mL or more.
18. The method of any one of embodiments 1-17, wherein the enriched population is bulk-cultured for at least about 4 days, at least about 5 days, or at least about 6 days, optionally, about 6 days.
19. The method of any one of embodiments 1-18, wherein the cells of the enriched population undergo at least about 7 cell divisions to yield the expanded IgG+ memory B cell population.
20. The method of any one of embodiments 1-19, wherein the cells of the expanded IgG+ memory B cell population are fused with myeloma cells by electrocell fusion (ECF) to obtain hybridomas.
21. The method of any one of embodiments 1-19, further comprising transferring the hybridomas and any unfused cells to selection medium, optionally, wherein the selection medium comprises HA.
22. The method of any one of embodiments 1-21, comprising storing hybridomas under freezing conditions.
23. The method of any one of embodiments 1-22, further comprising culturing hybridomas in multiplate wells and screening the supernatant of each well for antigen-specific antibodies.
24. A method of screening for hybridomas expressing antigen-specific antibodies, comprising
   a. generating hybridomas in accordance with any one of the methods of embodiments 1-23,
   b. culturing single hybridomas in individual wells; and
   c. screening the supernatant of each well for antigen-specific antibodies.
25. A method of screening for hybridomas expressing antigen-specific antibodies, comprising
   a. preparing an enriched population of IgG+ memory B cells from cells obtained from secondary lymphoid organs of one or more immunized non-human animals, wherein less than about 5% of the cells of the enriched population are IgM+ B cells;
   b. bulk-culturing the enriched population to obtain an expanded IgG+ memory B cell population;
   c. fusing cells of the expanded IgG+ memory B cell population with myeloma cells to obtain hybridomas;
   d. culturing single hybridomas in individual wells; and
   e. screening the supernatant of each well for antigen-specific antibodies.
26. A method of producing antigen-specific antibodies, comprising
   a. preparing an enriched population of IgG+ memory B cells from cells obtained from secondary lymphoid organs of one or more immunized non-human animals, wherein less than about 5% of the cells of the enriched population are IgM+ B cells;
   b. bulk-culturing the enriched population to obtain an expanded IgG+ memory B cell population;
   c. fusing cells of the expanded IgG+ memory B cell population with myeloma cells to obtain hybridomas;
   d. culturing single hybridomas in individual wells;
   e. screening the supernatant of each well for antigen-specific antibodies to identify the hybridomas expressing antigen-specific antibodies; and
   f. expanding the culture of the hybridomas identified in (e) to produce antigen-specific antibodies.
27. The method of any one of embodiments 1-1026, wherein less than about 3% of the cells of the enriched population are IgM+ B cells.
28. The method of embodiment 27, wherein less than about 2% of the cells of the enriched population are IgM+ B cells.
29. The method of embodiment 28, wherein less than about 1% of the cells of the enriched population are IgM+ B cells.

The following examples are given merely to illustrate the present invention and not in any way to limit its scope.

### EXAMPLES

### Example 1

This example describes an exemplary method of generating a hybridoma of the present disclosure.

Immunization

A cohort of 7 transgenic XenoMouse® G2-KL (XMG2-KL) animals (which are capable of producing human antibodies with either kappa or lambda light chains) were immunized with native Antigen Z stably expressed on CHO cells Animals were initially immunized with 4×10⁶ cells with adjuvant Alum/CpG ODN subcutaneously administered in the left thigh (SQ/LT), and then boosted twice weekly with 2×10⁶ cells for 8 weeks. A final boost was given 4 days before harvest of the animals and collection of spleen and draining lymph nodes.

Isolation & Enrichment

To isolate memory B cells, the spleen and lymph nodes (LNs) of the 7 immunized animals were harvested and processed to single cell suspensions (SCSs) per organ type: one SCS derived from the spleens of all animals and one SCS derived from the LNs of all animals Red blood cells (RBCs) were removed from the SCS derived from spleens using BD Pharm Lyse™ (BD Biosciences, Franklin Lakes, NJ). All SCSs were subsequently combined into one pooled SCS for further processing. B-cells were enriched using a 4-step enrichment procedure, wherein the first two steps removed IgM-positive (IgM+) cells, among other cells, and the last two steps positively selected from surface IgG-positive (IgG+) B cells.

In the first step, cells were incubated with a biotinylated antibody cocktail for 20 minutes at 4° C. The antibody cocktail comprised biotinylated antibodies that specifically bind to cell surface markers on non-B cells (e.g., T-cell, monocytes, macrophages, natural killer (NK) cells, granulocytes and RBCs) and human IgM-positive B-cells. The antibody cocktail included antibodies specific for NK1.1 (expressed by NK cells), IgM (expressed by IgM-positive B-cells), CD90.2 (expressed by T-cells), Ly-6G GR.1 (expressed by granulocytes and/or macrophages), CD3ε (expressed by T-cells), CD4 (expressed by T cells), CD8a (expressed by T-cells), CD11b (expressed by granulocytes, macrophages, dendritic cells, and/or NK cells) and TER119 (expressed by erythroid cells). After incubation with the cocktail, the cells were washed to remove unbound antibody. In the second step, streptavidin magnetic beads (e.g., Streptavidin Dynabeads (ThermoFisher Scientific, Pleasanton, CA) were incubated with the washed cells for 10 min at 4° C. to allow for the streptavidin magnetic beads to bind to the biotinylated antibodies which were bound to the non-B cells and the IgM-positive B-cells. A magnet was used to isolate and remove magnetic bead-labelled non-B cells and the IgM-positive B-cells. The remaining fraction of cells that were not bound to the magnetic beads contained the memory B-cell population, and, in the third step, this fraction was incubated with anti-human IgG antibody microbeads (Miltenyi, Bergisch Gladbach, Germany) for 40 min at 4° C. Finally, in the fourth step, the cells bound to the microbeads were applied to a magnetic column Surface IgG-positive cells were retained in the column, while surface IgG-negative cells passed through the column and collected for further processing. These surface IgG-positive cells representing memory cells were expelled or eluted from the column and then analyzed by FACS for surface expression of B220, IgG and IgM. Memory cells express high levels of B220 and IgG on the surface.

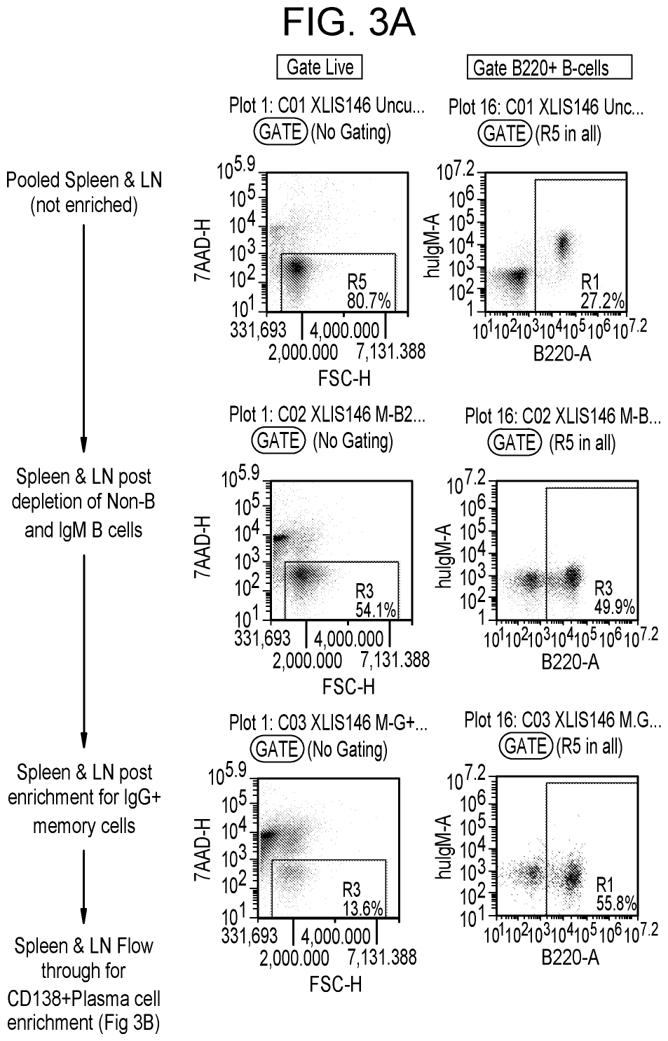

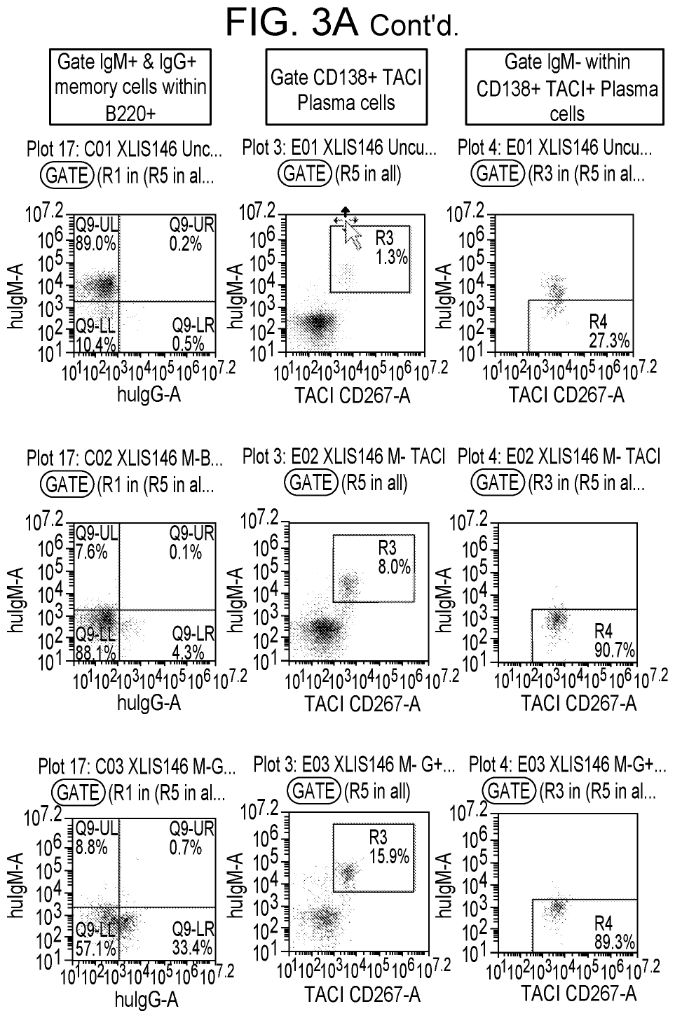

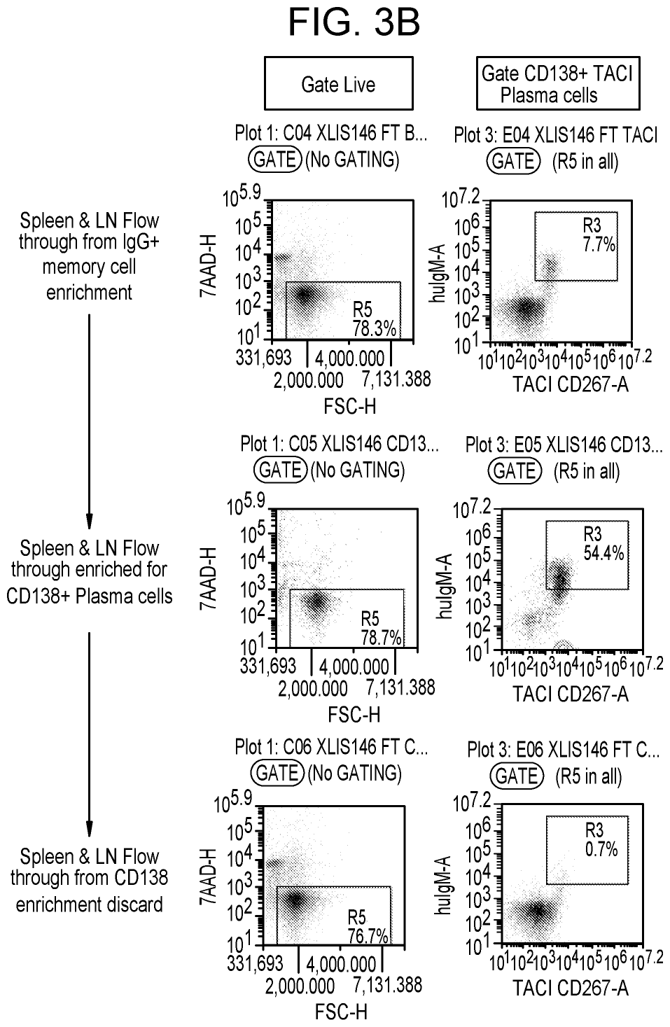

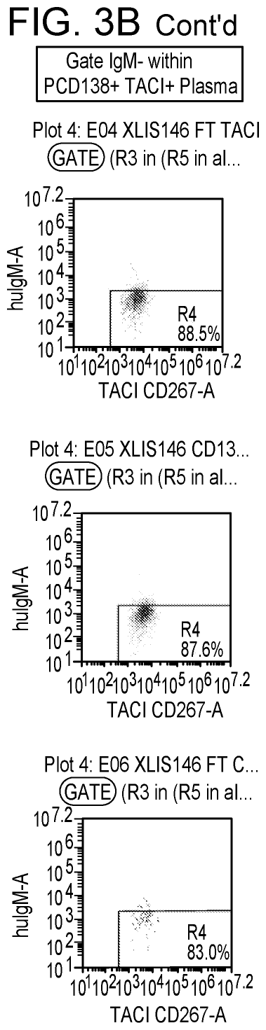

Exemplary FACS plots from the FACS analysis are shown in FIG. 3A. Table 1 provides a summary of the FACS analysis before, during and after the enrichment procedure for enriching IgG+ memory B cells. As shown in Table 1, the percentage of B220-positive cells substantially increased from 27.2% to 55.9% after the 4-step enrichment procedure. B220 is a cell surface marker of B cells (see, e.g., Khodadadi et al., Front Immunol 10: Article 721 (2019); doi: 10.3389/fimmu.2019.00721). After enrichment, as shown in Table 1, only 8.8% of the B220-positive cells were IgM-positive, while 33.4% of the B220-positive cells were IgG-positive. As shown in Table 1, the percentage of IgG+ cell of the total live cells increased from less than 1% to greater than 15%. Also, a substantial fraction (28.1%) of IgG+ B cells were recovered by this process (Table 1). The ratio of IgG+ cells to IgM+ cells increased from about 0.006 to about 3.8 (Table 1). The enrichment process increased this ratio over 600-fold as 99.96% of IgM+ B cells were removed by this enrichment process.

TABLE 1

| | | IgM− and | |
|---|---|---|---|
| | | Non-B cell | IgM−/IgG+ |
| | Uncut* | depleted** | enriched*** |
| Total live | 342,162,903 | 10,701,490 | 642,926 |
| % B220+ | 27.2 | 49.9 | 55.9 |
| % IgG+ of B220+ | 0.5 | 4.3 | 33.4 |
| % IgM+ of B220+ | 89.0 | 7.6 | 8.8 |
| Ratio of IgG+ cells | 0.006 | 0.57 | 3.8 |
| to IgM+ cells | | | |
| IgG+ count | 427,327 (<1.0%) | 228,950 (2%) | 120,074 (18%) |
| (% of total live) | | | |
| % Recovery of | 100 | 53.6 | 28.1 |
| IgG+ B cells | | | |
| % Depletion of | 0 | 99.51 | 99.96 |
| IgM+ B cells | | | |

*before enrichment process;
**after first and second steps of the B-cell enrichment procedure.
***after all four steps of the B-cell enrichment procedure.

The flow-through fraction containing the surface IgG-negative cells containing CD138/TACI double positive plasma cells was enriched in a separate procedure to rescue IgG secreting but not surface IgG-positive plasma cells via direct B cell discovery platforms. FACS was carried out to analyze the expression of B-cell markers to evaluate enrichment of IgM-negative plasma cells. Exemplary FACS plots from the FACS analysis of these cells are shown in FIG. 3B. This enriched fraction contains the high-affinity IgG secreting plasma cell population and is applied to direct B cell discovery technologies for antigen binding secretion assays.

Bulk Culture

The enriched population comprising IgG+ memory B cells obtained through the 4-step enrichment process were bulk cultured in T175 flasks at 625 B220-positive B cells/ml in RPMI media supplemented with FBS, gamma irradiated CD40L expressing EL4B5 feeder cell line, Rabbit T cell supernatant and human IgG cross linking microbeads. The volume of the bulk culture is generally about 200 mL. Cultures were incubated for 6 days at 37° C. at 5% CO 2. After bulk culturing, the cells were collected and counted. The post-bulk culture count was compared to the number of input B cells (number of B-cells used to inoculate the bulk culture) to calculate the number of cell divisions that took place during bulk culturing. Based on these counts, it was determined that the B cells underwent 9.8 cell divisions in bulk culture to produce an expanded population.

Cell Fusion

Fusion partner P3 myeloma cells were expanded and collected in log phase growth stage. The expanded population were combined with the P3 myeloma cells at a B-cell to P3 myeloma cell ratio of about 1:2.6. The cell mixture was washed twice in hypo-osmolar Electro Cell Fusion (ECF) buffer and resuspended to a density of 2×10⁶ cells/ml. Electro cell fusion was performed using a fusion chamber for high throughput fusion. In this experiment 150×10⁶ B cells were fused with 391×10⁶ P3 myeloma in 18 fusion events. Each fusion consists of 40 sec 60 v pre-alignment followed by 3×30 μsec pulses of 800V each.

Post-Fusion Culture and N2 Archiving

After cell fusion, the cells were immediately deposited into 270 ml DMEM media with FBS, washed once and placed into 3×200 ml T175 bulk cultures in DMEM with hypoxanthine azaserine (HA) to eliminate unfused myeloma cells. Following 3 days of post-fusion culture, the hybrid pool was collected, washed and split into 12 aliquots in 90% Newborn Calf Serum (NCS) & 10% DMSO for frozen storage. After 24 hrs at −80° C., the vials were transferred into liquid nitrogen for long term storage. One vial was thawed and plated in low density 96 well plates to evaluate clonal outgrowth in DMEM selection media. From here the fusion efficiency and the complexity of the pool was calculated. Here, after fusion, the total complexity was 205,882 unique hybrids and the fusion efficiency was calculated to be 0.140%.

Evaluation of Fusion Efficiency & Calculation of Complexity in Hybrid Pool

The maximal number of unique clones in a hybrid pool, coined “complexity”, is a calculated value that estimates what fraction of the enriched bulk cultured memory B cell population is immortalized in the hybrid pool. Combining complexity with the FACS informed recovery of memory B cells in the enrichment step enables approximation of the in-vivo immune repertoire which is captured in the EHG event. In this reduction to practice example, 28% of the total IgG+ memory B cells from the in-vivo repertoire was recovered after enrichment and these were entered into B cell culture and EHC. The fusion captured 60% of this fraction. Overall, 17% (28%*60%) of the IgG+ memory B cell repertoire from the pooled B cells of 7 animals was captured (immortalized) in this process.

Complexity calculations also guides the depth of interrogation of a hybrid pool. When plating hybrid pools, going beyond the calculated complexity is generally avoided as this increases the likelihood of repeated interrogation of clonal copies, while identifying new unique clones becomes increasingly less likely.

Calculation of complexity is based on 3 measures and 4 assumptions: The measures/variables were: (1) Enriched input number of B cells into culture; (2) Divisions in culture pre-fusion based on Day 6 count; and (3) Number of viable clones in low density plating of known volume from the resulting hybrid pool. The assumptions were: (1) Every enriched B cell submitted to bulk culture is a unique clone; (2) Every enriched B cell submitted to bulk culture survives 6 days of culture; (3) All enriched B cells submitted to bulk culture divides at the same rate; and (4) 50% of the fused cells are lost in freeze-thaw of the hybridoma pool.

Any Deviation from these assumptions reduces the complexity of the hybrid pool except for the freeze thaw, which can reduce or increase the complexity.

Complexity of a hybrid pool cannot exceed the input number of B cells into B cell culture

Complexity of hybrid pool=(Viable clones post-fusion after culture before freeze)/(2\hat{ }Divisions in culture post-fusion)

Viable clones post-fusion and culture, before freeze=(# of viable clone in low density seeding/50% loss in Freeze)/fraction of total hybrid pool evaluated for outgrowth

Divisions in culture post-fusion=1 division in the first 24 hrs, after that 1 division every 16 hours=1+(# days in culture post-fusion−1)*(24/16)

Divisions in B cell culture=LOG(Day 6 Live cells/Day 0 Input B cells)/LOG(2)

Clonal copies after B cell culture=2\hat{ }Div in Culture

Clonal copies after fusion=Clonal B cell copies at Day 6 of culture*% Fusion efficiency

% Fusion efficiency=Complexity/Day 6 B cells for fusion

If the number of clonal copies after fusion is >1 (every clone represented more than once) then the formula for clonal copies frozen after post fusion culture is adjusted to account for siblings.

### Example 2

This example describes another example of generating hybridomas using the method of the present disclosure.

A cohort of 6 XenoMouse® animals (four XenoMouse® G2-K (XMG2-K) animals which are capable of producing human antibodies with kappa light chains; and two XenoMouse® G4-KL (XMG4-KL) animals which are capable of producing human antibodies with either kappa or lambda light chains)) were immunized with Antigen X, which was a different antigen from Antigen Z of Example 1 Animals were initially immunized with Antigen X expressed by CHO-S cells administered intraperitoneally (IP) and then boosted twice weekly for 6 weeks. Mice were dormed for 2.5 months. A final boost was given and 4 days later the spleen and draining lymph nodes (LN) from the immunized mice were harvested. As in Example 1, harvested spleens were pooled together and processed into a SCS and harvested draining LNs were pooled together and processed into a SCS. RBCs were removed from the one SCS derived from spleens, as essentially described in Example 1, and then combined with the SCS derived from LNs to obtain a pooled SCS for further processing.

In Example 1, a four-step B-cell enrichment process is described and was followed by bulk culturing. The first and second steps of the B-cell enrichment process were purposed for depletion of IgM-positive (IgM+) cells while the last two steps were purposed for enrichment for surface IgG+ cells (through positive selection of surface IgG+ cells using anti-human IgG antibody-labeled magnetic beads). To evaluate the importance of the steps of the B-cell enrichment process and the bulk culturing on hybridoma generation, the pooled SCS was split into 5 groups (Groups 1-5) wherein each group was subjected to a unique protocol and varied by including or excluding the IgG+ enrichment and including or excluding the bulk culturing. A summary of the treatment of each of the 5 groups is provided in Table 2.

TABLE 2

| | Depletion | Enrichment | Bulk |
|---|---|---|---|
| | of IgM+ | for Surface | Culturing |
| | cells* | IgG+ cells** | for 6 days |
| Group 1 | − | − | − |
| Group 2 | + | − | − |
| Group 3 | − | − | + |
| Group 4 | + | − | + |
| Group 5 | + | + | + |

*First two steps of the 4-step enrichment process as described in Example 1 (which included depletion of non-B cells)
**Last two steps of the 4-step enrichment process as described in Example 1

As shown in Table 2, Groups 1 and 2 lacked any bulk culturing. Group 1 additionally was not subjected to any steps of the enrichment process and were considered “uncut”, whereas Group 2 was subjected to only the first two steps of the 4-step enrichment process as described in Example 1. Groups 3-5 were bulk cultured for 6 days but only Group 5 included both an IgM+ cell depletion (as achieved by the first two steps of the enrichment process described in Example 1) and a surface IgG+ cell enrichment (as achieved by the last two steps of the enrichment process described in Example 1), whereas Group 3 excluded both the IgM+ cell depletion and surface IgG+ cell enrichment, and Group 4 excluded the surface IgG+ cell enrichment. The IgM+ cell depletion of Groups 2, 4, and 5 was carried out as essentially described in Example 1. The surface IgG+ cell enrichment of Group 5 was carried out as essentially described in Example 1. A summary of characteristics of each of Groups 1-5 (pre-bulk-culture) are provided in Table 3.

TABLE 3

| | % Recovery of | Ratio of | % IgG | % IgM | % Depletion |
|---|---|---|---|---|---|
| | IgG+ B cells | IgG:IgM of | fraction of | Fraction of | of IgM+ |
| | of Enriched | Enriched | Enriched | Enriched | B cells |
| | Population | Population | population | Population | achieved |
| Group 1 | 100 | 0.019 | 0.59 | 31.3 | 0 |
| Group 2 | 46 | 0.893 | 6.7 | 7.5 | 99.02 |
| Group 3 | 100 | 0.019 | 0.59 | 31.5 | 0 |
| Group 4 | 46 | 0.893 | 6.7 | 7.5 | 99.02 |
| Group 5 | 17 | 4.673 | 37.6 | 8.0 | 99.93 |

As shown in Table 3, for Groups 2, 4, and 5 (which were subjected to at least part of the 4-step enrichment process), less than or about 10% of the enriched population are IgM-positive (IgM+) B cells. Also for Group 5 (which was subjected to all steps of the enrichment process), the ratio of the IgG+ memory B cell count to IgM+ B cell count of the enriched population is greater than, meaning that the number of IgG+ cells outnumbered the IgM+ cells.

The cells of Groups 3-5 were bulk cultured as essentially described in Example 1 to obtain an expanded population. The cells of Groups 1-5 were used for cell fusion, which was carried out as described in Example 1. Following fusion, the cells were cultured for 3 days in DMEM with HA. Post-fusion, live cells were counted and screened for secretion of Antigen-X-specific antibodies using FMAT fluorometric microvolume assay technology (FMAT™ 8100 HTS System).

The results of the count and characterization are provided in Table 4.

TABLE 4

| | | | # of 96-well | Relative % | % of the |
|---|---|---|---|---|---|
| | | | plates required | antigen- | IgG+ memory |
| | | | for full | specific | B cell |
| | Complexity | % hit | interrogation | repertoire | repertoire |
| Group | of hybrid pool | frequency | of repertoire | captured | captured |
| 1 | 24,479 | 0.62 | 52 | 0.61 | 0.05 |
| 2 | 332 | 2.03 | 1 | 0.03 | 0.01 |
| 3 | 19,131,781 | 0.13 | 39,858 | 100 | 100 |
| 4 | 69,993 | 8.00 | 146 | 23 | 6.3 |
| 5 | 51,093 | 14.90 | 109 | 31 | 10.8 |

Complexity of hybrid pool represents (viable clones post-fusion after culture before freeze)/(2\hat{ }Divisions in culture post-fusion).
% hit frequency represents the number of antigen-specific (Ag+) hybridoma clones/total hybridoma clones screened.
# 96-well plates required for full interrogation of repertoire represents the total number of 96-well plates required to seed the full complexity of the hybridoma pool at 5 clones/well or 480 clones/plate. The plating is followed by screening for antigen specific binding.
Relative % antigen-specific repertoire captured represents the percentage of antigen-specific cells recovered after hybridoma generation; it is the normalized expression of Ag+ hybrid clones generated using each of the 5 methods. For each method, starting with the same number of input live cells, the number of Ag+ binding clones is calculated as = complexity * % Ag+ hit frequency. This number is then normalized to a percentage of the maximal number of Ag+ clones obtained (method 3).
% of the IgG+ memory B cell repertoire captured is the calculated fraction of IgG+ B that were successfully immortalized taking into account losses during enrichment and fusion

As shown in Table 4, Groups 1 and 2 (which were not bulk cultured) resulted in the lowest # of hybridoma clones generated and the lowest % of the antigen+repertoire captured, which results support the importance of bulk culturing. Among Groups 3-5 (which were bulk cultured), the % hit frequency was highest for Groups 4 and 5 (which were IgM depleted before bulk culturing). The % hit frequency almost doubled when both IgM depletion and surface IgG enrichment was carried out (compare Group 4 to Group 5). Taken together, these results demonstrate the advantages of bulk culturing a B-cell enriched population prior to cell fusion. The relative % antigen+repertoire captured by Group 5 was calculated as 31% (Table 4). Given that some B-cells are inevitability lost during purification and that B-cells in a germinal centre contain sisters and duplicate specificities, the 31% repertoire captured by Group 5 is excellent and likely represents the full compartment. Additionally, that only 109 96-well plates would be needed to interrogate the full repertoire (at a 14.9% hit frequency), interrogation of the full repertoire is feasible, which is not true for the method of Group 3.

### Example 3

This example describes a third example of generating a hybridomas using the method of the present disclosure.

The presently disclosed method of generating hybridomas was used to generate hybridomas which produce high affinity, antigen-specific antibodies. In this example, the antigen was a G-Protein Coupled Receptor (GPCR). GPCRs constitute a therapeutically relevant target class that is notoriously challenging for targeting with antibodies (Hutchings C J, Expert Opin. Biol. Ther. 2020, vol 20, No. 8, 925-935). While antibodies to this GPCR have been made before, none have been able to cross react with both the human and cynomolgus monkey orthologs, let alone have an affinity of at least 1 nM for each ortholog. Thus, it was a goal to generate hybridomas which secrete human/cyno cross-reactive, GPCR-specific antibodies exhibiting an affinity for antigen of at least 1 nM for each of the human and cynomolgus monkey ortholog.

Over 300 Xenomouse animals (e.g., XenoMouse® G2-K (XMG2-K) animals which are capable of producing human antibodies with kappa light chains) were immunized with the GPCR antigen following an immunization campaign comprising GPCR DNA immunization (via gene gun), peptides spanning the extracellular regions of the GPCR, GPCR transfected cells, and extracellular domains of the GPCR fused on human IgG-Fc portion. Following immunization, sera from each of immunized animal was collected and evaluated for GPCR-specific antibody titer using standard methods (e.g., evaluation of polyclonal antibody binding by FACS analysis on GPCR transiently expressed on 293T cells). Results of the antibody titer analysis revealed that 48 animals (15% of the total number immunized) exhibited a sufficient level of antigen-specific antibody titer, as determined by an at least 3-fold higher binding GeoMean signal on GPCR transfected cells as compared to GeoMean signal on mock transfected cells.

In this example, hybridomas generated from the secondary lymphoid organs of one of these 48 animals is described. Briefly, four days after the last immunization boost, the spleens and draining LN of the animal were harvested. An SCS was prepared from the spleen and a separate SCS was prepared from the LNs. RBCS were depleted from the SCS prepared from the spleen as described in Example 1, and then the RBC-depleted SCS was combined with the SCS prepared from the LNs. This combined SCS was subjected to only the first two steps of the four-step B-cell enrichment process described in Example 1. IgM+ cell/non-B cell depletion reduced live cell count by 99.5% and reduced the IgM+ B cells by 99.8%. The ratio of IgG to IgM increased 70-fold. The % B220+ cells of the enriched population increased to 46.2% after the IgM+ cell/non-B-cell depletion. Also, the % IgG+ cells of the B220+ cells increased to 7.5% after the IgM+ cell/non-B-cell depletion. After IgM and Non-B-cell depletion, at least 10% of the IgG+ cells were recovered.

Approximately 40,000 B-cells were bulk cultured, as described in Example 1, and the bulk culturing process resulted in about 11 cell divisions. Following bulk culturing, the cells were subjected to cell fusion as in Example 1. After B-cell bulk culture and fusion, an estimated 40% of the cultured B cells were immortalized generating a hybrid pool with a complexity of 16,000 unique hybrids. Post-fusion, cells were cultured in DMEM with HA to eliminate unfused myeloma cells. Subsequently, hybridoma cells were single cell sorted into 384-well plates on soluble antigen recapitulating specific regions of GPCR of interest or plated polyclonally in 96-well plates at 5 hybridoma clones per well. Hybridoma cells were cultured to produce sufficient antibody to detect in FACS-based screening. Screening was carried out by FACS analysis, by determining antibody binding in culture supernatant to 293T cells transiently transfected with the GPCR or by binding to a cancer cell line endogenously expressing the GPCR. This is the same procedure that was used in Example 1 (except here 293T cells expressing GPCR antigen were used). Characterization of binding to human and cyno orthologs was also carried out. The affinity of the final selected molecules was determined by on-cell KinExa.

Over the course of several months to years, hybridomas using the secondary lymphoid organs of the remaining animals selected based on antibody titer analysis (the remaining 47 animals) were generated in similar fashion to that described above. More than 4000 hybridoma clones producing GPCR-specific antibodies were identified across the 48 immunized. However, only one had an affinity>1 nM to both human and cyno GPCR orthologs. Taken together, these results demonstrate the remarkable deep repertoire mining power of the presently disclosed EHG methods. Given the top challenge around GPCRs as a target class and the high difficulty affinity design goal of <1 nM for both human and cyno orthologues, the expectation was that this antibody will be very rare. This method enabled the interrogation of the immune repertoires of 48 antigen-responding animals, recovery of >4000 binders, and identification of a single clone meeting design goals.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of generating hybridomas, comprising
a. preparing an enriched population of IgG-positive (IgG+) memory B cells from cells obtained from secondary lymphoid organs of one or more immunized non-human animals, wherein:
i. less than or about 10% of the enriched population are IgM-positive (IgM+) B cells, and/or
ii. the ratio of the IgG+ memory B cell count to IgM+ B cell count of the enriched population is greater than 0.5, optionally, greater than 1 or greater than 2;
b. bulk-culturing the enriched population to obtain an expanded population; and
c. fusing cells of the expanded population with myeloma cells to obtain hybridomas.

2. The method of claim 1, further comprising (a) immunizing one or more non-human animal(s) with an immunogen, (b) harvesting secondary lymphoid organs from the immunized non-human animal(s), optionally, about 3 to about 5 days post-immunization, (c) preparing a single-cell suspension (SCS) from the secondary lymphoid organs harvested from each immunized non-human animal, and/or (d) preparing a pooled SCS by combining the SCS from the secondary lymphoid organs of more than one immunized non-human animals.

3. The method of claim 1 or 2, wherein the enriched population of IgG+ memory B cells are prepared from a single-cell suspension of cells obtained from the secondary lymphoid organs.

4. The method of any one of the preceding claims, wherein the secondary lymphoid organs are secondary lymphoid organs harvested from the immunized non-human animal(s) about 3 to about 5 days post-immunization.

5. The method of any one of the preceding claims, wherein the secondary lymphoid organs are secondary lymphoid organs harvested from at least 1, 2, 3, 4, or 5 immunized non-human animal(s).

6. The method of any one of the preceding claims, wherein the secondary lymphoid organs are spleen and/or lymph nodes.

7. The method of any one of the preceding claims, wherein the secondary lymphoid organs are the secondary lymphoid organs harvested from only select immunized non-human animals, optionally, wherein the select immunized non-human animals were selected based on post-immunization serum antibody titer level.

8. The method of any one of the preceding claims, wherein the non-human animals are mice or rats.

9. The method of any one of the preceding claims, wherein the enriched population is prepared by removing IgM+ cells and/or selecting for IgG+ cells.

10. The method of any one of the preceding claims, wherein the enriched population is prepared by a negative selection of IgM+ cells and/or a positive selection for IgG+ cells.

11. The method of claim 10, wherein greater than 90% IgM+ B cells are removed upon the negative selection and/or the positive selection, optionally, wherein greater than 95% IgM+ B cells are removed upon the negative selection and/or the positive selection.

12. The method of claim 11, wherein greater than 98% IgM+ B cells are removed upon the negative selection, optionally, wherein greater than 99% IgM+ B cells are removed upon the negative selection and the positive selection.

13. The method of any one of the preceding claims, wherein the enriched population is prepared by removing non-B cells, red blood cells (RBCs), IgM+ cells, or a combination thereof, from a single cell suspension prepared from the secondary lymphoid organs, optionally, comprising removing non-B cells, RBCs, IgM+ cells, or a combination thereof, using antibodies specific to one or more cell surface markers expressed by the non-B cells, RBCs, or IgM+ cells.

14. The method of claim 13, wherein the cell surface markers are human IgM, CD90.2, Ly-6G GR.1, NK-1.1, CD3epsilon, CD4, CD8a, CD11b, and/or TER119.

15. The method of claim 13 or 14, wherein the antibodies are linked to biotin and the method comprises using streptavidin-labeled beads, optionally, streptavidin-labeled magnetic beads, to remove the non-B cells, RBCs, and/or IgM+ cells.

16. The method of claim 15, wherein greater than 98% of the IgM+ cells present in the single cell suspension are removed.

17. The method of any one of the preceding claims, wherein the enriched population is prepared by selecting for surface IgG+ cells, optionally, by using anti-IgG antibody-labeled beads, optionally, anti-human IgG antibody-labeled magnetic beads.

18. The method of claim 17, wherein greater than 99% of the IgM+ cells present in the single cell suspension are removed and/or wherein the ratio of the IgG+ memory B cell count to IgM+ B cell count increases by at least about 50-fold or at least about 100-fold.

19. The method of any one of the preceding claims, comprising bulk-culturing the enriched population of IgG+ cells with anti-human IgG antibody-labeled beads and feeder cells in a cell culture medium comprising rabbit T-cell supernatant.

20. The method of claim 19, wherein the feeder cells express CD40L and/or are gamma-irradiated.

21. The method of claim 20, wherein the feeder cells are gamma-irradiated, CD40L-positive EL4B5 feeder cells.

22. The method of any one of the preceding claims, wherein the myeloma cells are in a log phase growth stage.

23. The method of any one of the preceding claims, wherein the myeloma cells are P3 myeloma cells.

24. The method of any one of the preceding claims, wherein bulk-culturing the enriched population is initiated with a seeding density of about 350 B220-positive B cells per mL to about 700 B220-positive B cells, optionally, about 600 B220-positive cells per mL to about 650 B220-positive cells per mL.

25. The method of any one of the preceding claims, wherein the enriched population is bulk-cultured in a volume of at least about 25 mL and less than or about 2 L.

26. The method of claim 25, wherein the enriched population is bulk-cultured in a volume of about 50 mL to about 500 mL, optionally, about 100 mL to about 300 mL.

27. The method of any one of the preceding claims, comprising bulk-culturing the enriched population for at least about 4 days, at least about 5 days, or at least about 6 days.

28. The method of claim 27, comprising bulk-culturing the enriched population for at least about 5 days.

29. The method of claim 28, comprising bulk-culturing the enriched population for about 6 days.

30. The method of any one of the preceding claims, wherein the cells of the enriched population undergo at least about 6 or at least about 7 cell divisions to yield the expanded population.

31. The method of any one of the preceding claims, wherein all B cells of the expanded population are used for fusing with myeloma cells and/or all cells of the expanded population are combined with myeloma cells.

32. The method of any one of the preceding claims, wherein B cells are not selected for fusing with myeloma cells based on production of antibodies which bind to an antigen and/or B cells of the enriched population or the expanded population are not screened for production of antibodies which bind to an antigen.

33. The method of any one of the preceding claims, wherein the cells of the expanded population are fused with myeloma cells by electrocell fusion (ECF) to obtain hybridomas, optionally, wherein cells of the expanded population are fused with myeloma cells in a volume greater than 10 mL per fusion event.

34. The method of any one of the preceding claims, further comprising transferring the hybridomas and any unfused cells to selection medium, optionally, wherein the selection medium comprises hypoxanthine azaserine (HA).

35. The method of any one of the preceding claims, further comprising storing hybridomas under freezing conditions.

36. The method of any one of the preceding claims, further comprising screening the hybridomas for production of antibodies which bind to an antigen.

37. The method of any one of the preceding claims, further comprising culturing hybridomas in multiplate wells and screening the supernatant of each well for antigen-specific antibodies.

38. The method of claim 36 or 37, wherein the screening comprises an immunoassay which detects binding of antibodies to the antigen.

39. The method of claim 38, wherein the immunoassay is a fluorescence activated cell sorting (FACS) analysis.

40. The method of any one of the preceding claims, wherein screening for cells producing antigen-specific antibodies occurs only after hybridomas are obtained, and not before hybridomas are obtained.

41. The method of any one of the preceding claims, wherein screening for antigen-specific antibodies occurs only before secondary lymphoid organs are harvested and after hybridomas are obtained.

42. The method of claim 41, wherein the screening for antigen-specific antibodies that occurs before secondary lymphoid organs are harvested comprises a titer analysis of serum obtained from live immunized animals.

43. A method of generating hybridomas producing antigen-specific antibodies, comprising
a. immunizing one or more non-human animal(s) with an immunogen;
b. harvesting secondary lymphoid organs from the immunized non-human animal(s);
c. preparing a single-cell suspension (SCS) from the secondary lymphoid organs harvested from each immunized non-human animal;
d. preparing a pooled SCS by combining all SCSs prepared in (c);
e. removing greater than 95% IgM+ cells from the pooled SCS and/or positively selecting for surface IgG+ cells from the pooled SCS to obtain an enriched population of IgG-positive (IgG+) memory B cells, wherein:
i. less than or about 10% of the enriched population are IgM-positive (IgM+) B cells and/or
ii. the ratio of the IgG+ memory B cell count to IgM+ B cell count of the enriched population is greater than 0.5, optionally, greater than 1 or greater than 2,
f. bulk-culturing the enriched population to obtain an expanded population;
g. fusing cells of the expanded population with myeloma cells to obtain hybridomas; and
h. identifying the hybridomas producing antigen-specific antibodies by culturing single hybridomas in individual wells and screening the supernatant of each well for antigen-specific antibodies.

44. A method of screening for hybridomas expressing antigen-specific antibodies, comprising
a. generating hybridomas in accordance with any one of the methods of any one of the preceding claims,
b. culturing single hybridomas in individual wells; and
c. screening the supernatant of each well for antigen-specific antibodies.

45. A method of screening for hybridomas expressing antigen-specific antibodies, comprising
a. preparing an enriched population of IgG+ memory B cells from cells obtained from secondary lymphoid organs of one or more immunized non-human animals, wherein less than about 5% of the cells of the enriched population are IgM+ B cells;
b. bulk-culturing the enriched population to obtain an expanded population;
c. fusing cells of the expanded population with myeloma cells to obtain hybridomas;
d. culturing single hybridomas in individual wells; and
e. screening the supernatant of each well for antigen-specific antibodies.

46. A method of producing antigen-specific antibodies, comprising
a. preparing an enriched population of IgG+ memory B cells from cells obtained from secondary lymphoid organs of one or more immunized non-human animals;
b. bulk-culturing the enriched population to obtain an expanded population;
c. fusing cells of the expanded population with myeloma cells to obtain hybridomas;
d. culturing single hybridomas in individual wells;
e. screening the supernatant of each well for antigen-specific antibodies to identify the hybridomas expressing antigen-specific antibodies; and
f. expanding the culture of the hybridomas identified in (e) to produce antigen-specific antibodies.

47. The method of any one of the preceding claims, wherein the hybridomas obtained represent at least 15% of the IgG+ memory B cell repertoire produced by the immunized animals.

48. The method of claim 44, wherein the hybridomas obtained represent at least 10% of the IgG+ memory B cell repertoire produced by the immunized animals, optionally, at least 15% of the IgG+ memory B cell repertoire produced by the immunized animals.

49. The method of claim 48, wherein the hybridomas obtained represent at least 20% of the IgG+ memory B cell repertoire produced by the immunized animals.

50. The method of claim 49, wherein the hybridomas obtained represent at least 25% of the IgG+ memory B cell repertoire produced by the immunized animals.
